(19) United States
(12) Patent Application Publication
(10) Pub. No.: US 2004/0014946 A1
(43) Pub. Date: Jan. 22, 2004

(54) Protein interaction method and composition

(76) Inventors: Heman Chao, Aurora (CA); Wah Y. Wong, Edmonton (CA); Baomin Tian, Edmonton (CA); Donald Segal, Stouffville (CA); Jerry McElroy, Richmond Hill (CA)

Correspondence Address: PERKINS COIE LLP, P.O. BOX 2168, MENLO PARK, CA, 94026, US

(21) Appl. No.: 10/422,561

(22) Filed: Apr. 23, 2003

Related U.S. Application Data
(60) Provisional application No. 60/375,627, filed on Apr. 25, 2002.

(51) Int. Cl. G01N 33/567; G01N 33/53; C07K 14/72; C07K 14/705; B05D 3/00

(52) U.S. Cl. 530/350; 435/7.2; 427/2.11; 530/358

(57) ABSTRACT

A protein interaction method and composition are described. The method and composition are useful for a number of purposes including: reconstituting multisubunit protein complexes, identifying known binding subunits, determining the kinetics and order of self assembly of a multisubunit protein complex, and drug screening. The method involves contacting a conjugate with a solid surface having an immobilized first coil-forming peptide characterized by a selected charge and an ability to interact with a second, oppositely charged coil-forming peptide to form a stable α-helical coiled-coil heterodimer, where the conjugate comprises (a) the second, oppositely charged coil-forming peptide, and (b) a first subunit polypeptide which is one of a plurality of subunit polypeptides in a multisubunit complex. By said contacting the conjugate is bound to the solid surface. Other subunits of the complex are added under conditions effective to promote self-assembly of the subunit complex on the solid surface.

This application claims the benefit of U.S. Provisional Application No. 60/375,627, filed Apr. 25, 2002, which is incorporated herein by reference in its entirety.

### FIELD OF THE INVENTION

The present invention relates to methods for identification and analysis of multisubunit protein complexes, and to biofunction chips and compositions for use in practicing the method.

### REFERENCES

Abrahmsen et al., (1991) Biochemistry, 30:4151.

Aranda, A and Pascual, A. (2001) Nuclear hormone receptors and gene expression. Physiol Rev 81: 1269-1304.

Bressers et al., (1996) J Electro-Analytical Chem, 406:131.

Castillo, Al et al. (1999) Synergistic activation of the prolactin promoter by vitamin D receptor and GHF-1: role of the coactivators, CREB-binding protein and steroid hormone receptor coactivator-1 (SRC-1). Mol Endocrinol 13: 1141-1154.

Catterall, W. A. Structure and function of voltage-gated ion channels. Annu. Rev. Biochem. 64, 493-531 (1995).

Cavailles, V et al. (1995) Nuclear factor RIP140 modulates transcriptional activation by the estrogen receptor. EMBO J 14: 3741-3751.

Clark-Lewis et al., (1987) FEBS Lett 307:97.

Clark-Lewis et al., (1994) J Biol Chem 269:16075.

Clark-Lewis et al., (1991) Biochemistry 30:3128.

Dawson et al., (1994) “Synthesis of Proteins by Native Chemical Ligation,” Science 266:776-779.

Fang, G., Yu, H., and Kirschner, M. W. (1998) Direct binding of CDC20 protein family members activates the anaphase-promoting complex in mitosis and G1. Mol Cell 2,163-71.

Hershko, A. and Ciechanover, A. (1998) The ubiquitin system. Annu. Rev. Biochem. 67, 425-479.

Ideue, S. et al., (2000) Chemical Physics Letters, 337:79-84.

Janshoff et al., (1998) J Am Chem Soc 120:12108.

Karin, M., and Ben-Neriah, Y. (2000). Phosphorylation meets ubiquitination: the control of NF-kappaB activity. Annu Rev Immunol 18, 621-63.

Lee et al., (1995) Mater Res Soc Symp Proc 338:125.

deLisle Milton et al., (1992) “Techniques in Protein Chemistry IV,” Academic Press, New York, pp. 257-267.

Maniatis, T. (1999). A ubiquitin ligase complex essential for the NF-kappaB, Wnt/Wingless, and Hedgehog signaling pathways. Genes and Development 13, 505-10.

Petrova-Koch et al., (1992) Appl Phys Lett 61:943.

Polakis, P. (1999). The oncogenic activation of beta-catenin. Curr Opin Genet Dev 9, 15-21.

Rajarathnam et al., (1994) Biochemistry 29:1689.

Ribeiro, RC et al., (1992) Thyroid hormone alters in vitro DNA binding of monomers and dimers of thyroid hormone receptors. Mol Endocrinol 6: 1142-1152.

Schnolzer et al., (1992) Science 256:221.

Schulman, IG et al., (1995) Interactions between the retinoid X receptor and a conserved region of the TAT-binding protein mediate hormone-dependent transactivation. Proc natl Acad Sci USA 92: 8288-8292.

Varshavsky, A. (1997) The ubiquitin system. Trends Biochem Sci 22, 383-7.

Whittal et al., (1998) Anal Chem 70:5344-5347.

Wilson, CJ et al. (1996) RNA polymerase II holoenzyme contains SWI/SNF regulators involved in chromatin remodeling. Cell 84: 235-244.

Yamano, H., Tsurumi, C., Gannon, J., and Hunt, T. (1998). The role of the destruction box and its neighbouring lysine residues in cyclin B for anaphase ubiquitin-dependent proteolysis in fission yeast: defining the D-box receptor. Embo J 17, 5670-8.

Zachariae, W., and Nasmyth, K. (1999) Whose end is destruction: cell division and the anaphase-promoting complex. Genes Dev 13, 2039-58.

Zachariae, W., Schwab, M., Nasmyth, K., and Seufert, W. (1998) Control of cyclin ubiquitination by CDK-regulated binding of HctI to the anaphase promoting complex. Science 282, 1721-4.

### BACKGROUND OF THE INVENTION

A number of protein expression systems have been used as tools in biochemical research. These expression systems include genetically engineered cell lines that over-express a protein of interest (e.g. receptor, antibody or enzyme) modified bacteria, and phage display libraries of multiple proteins. Proteins prepared through these approaches can be isolated and either screened in solution or attached to a solid support for screening against a target of interest such as other proteins, receptor ligands, small molecules, and the like. Recently, a number of researchers have focused their efforts on the formation of arrays of proteins similar in concept to the nucleotide biochips currently being marketed. For example, WO 00/04389 and WO 00/04382 describe microarrays of proteins and protein-capture agents formed on a substrate having an organic thinfilm and a plurality of patches of proteins, or protein-capture agents. Also, WO 99/40434 describes a method of identifying antigen/antibody interactions using antibody arrays and identifying the antibody to which an antigen binds.

While arrays of proteins, and protein-capture agents provide a method of analysis distinct from nucleotide biochips, the preparation of such arrays requires purification of the proteins used to generate the array. Additionally, detection of a binding or catalytic event at a specific location requires either knowing the identification of the applied protein, or isolating the protein applied at that location of the array and determining its identity. Also, attachment of proteins to an array sometimes causes these proteins to lose their ability to interact with other proteins or ligands after immobilization.

What is needed is a means to identify protein binding events wherein the protein is presented to the binding agent or substrate in a state in which it retains the ability to interact with other proteins. Additionally, it would be preferable to have the protein presented in a manner that allows for efficient isolation and/or identification of the proteins for which binding or catalytic events are detected. Finally, the system should enable rapid analysis of the proteins by coupling of the arrays to detection systems that allow for the rapid, high-throughput analysis of chemical or biological samples. Such techniques would be extremely valuable in identifying subunits in multisubunit complexes. The present invention is designed to meet these needs.

### SUMMARY OF THE INVENTION

In one aspect, the invention includes a protein interaction method. In practicing the method, a conjugate is contacted with a solid surface having an immobilized first coil-forming peptide characterized by a selected charge and an ability to interact with a second, oppositely charged coil-forming peptide to form a stable α-helical coiled-coil heterodimer. The conjugate includes the second, oppositely charged coil-forming peptide, and a first subunit polypeptide which is one of a plurality of subunit polypeptides in a multisubunit complex. This allows the conjugate to bind to the solid surface. Other subunits of the complex are then added to the solid surface and bound first subunit polypeptide under conditions effective to promote self-assembly of the subunit complex on the solid surface.

In one embodiment, the first subunit polypeptide is a nuclear hormone receptor. Preferably, the nuclear hormone receptor is selected from the group consisting of androgen receptors, thyroid receptors, estrogen receptors, vitamin D receptors, retinoic acid receptors, glucocorticoid receptors, and mineralocorticoid receptors.

The method is readily adapted for use in reconstituting a multiprotein complex of known formulation, where the other subunits are added individually. Alternatively, the method may be used for identifying unknown binding subunits, where the other subunits are added as a mixture.

In another embodiment, the method is used for isolating a multisubunit protein complex from a host cell, wherein the other proteins in the multisubunit complex are pre-assembled and contained within the host cell. This method includes lysing the host cells prior to adding the other proteins to the solid support, and analyzing the multisubunit complex after adding the remaining subunits to determine the subunits constituting the multiprotein complex. The host cell, in one embodiment, is a diseased cell. Alternatively, the host cell is a normal cell. The host cell may be treated with an agent selected from the group consisting of hormones, ligands, and drugs.

In one embodiment, the method is useful for determining the kinetics and/or order of self assembly of a multisubunit protein complex. This embodiment includes the steps of analyzing the subunits bound to the solid support at various times after the addition of the other subunits, and determining the rate or order of subunit assembly of the protein complex.

In another embodiment, the method is useful for drug screening. This embodiment includes the steps of contacting the solid surface with one or more chemical compounds under conditions effective to allow the compounds to bind to the self-assembled multisubunit protein complex, washing the solid surface to remove unbound components, and analyzing the complex to identify the bound compounds.

The surface, in one embodiment of the invention, is a modified target plate suitable for MALDI mass spectrometry.

In yet another embodiment, the invention includes a method for carrying out the interaction of a plurality of multisubunit protein complexes. In practicing the method, to each of a plurality of wells in a substrate, each well having a first coil-forming peptide therein, adding a selected one of a plurality of different-sequence subunit molecules, each having a common second coil-forming peptide capture portion and a different-sequence target protein portion selected from a plurality of subunits in a multisubunit protein complex. The wells are contacted with the remaining subunits from each of the multisubunit protein complexes under conditions effective to promote self-assembly of each of the complexes. The wells are then washed to remove unbound components.

In another aspect, the invention includes a composition comprising a first coil-forming peptide having a selected charge and capable of interacting with a second, oppositely charged coil-forming peptide to form a stable a-helical coiled-coil heterodimer immobilized on a solid surface. A protein conjugate is bound to the first coil-forming peptide. The protein conjugate includes the second, oppositely charged coil-forming peptide, and a first target subunit selected from a plurality of subunits in a multisubunit protein complex. Other subunits of the complex are assembled on the solid surface through protein interactions with the protein conjugate.

In yet another aspect of the invention includes biofunction chip for measuring the activity of a first or second biomolecule. The chip includes a surface containing a plurality of spatially discrete regions, wherein each region is functionalized with a first coil-forming peptide having a selected charge and interacts with a second, oppositely charged coil-forming peptide to form a stable a-helical coiled-coil heterodimer. The first biomolecule is attached to the distal end of the second coil-forming peptide. Interaction of the first biomolecule with the second biomolecule is effective to modify the first or second biomolecule or both.

In one embodiment, each region on the chip comprises a plurality of first coil-forming peptides.

In another embodiment, the first biomolecule is selected from the group consisting of proteins, glycoproteins, natural and synthetic peptides, alkaloids, polysaccharides, nucleic acid molecules, and small molecules In a related embodiment, the second biomolecule is selected from the group consisting of proteins, glycoproteins, natural and synthetic peptides, alkaloids, polysaccharides, nucleic acid molecules, and small molecules.

In another embodiment, the first biomolecule is selected from the group consisting of a kinase substrate, a histone acetyl transferase substrate, and a protease substrate. In a preferred embodiment, the first biomolecule is a nucleic acid molecule. In a related embodiment, the nucleic acid molecule is modified by methylation.

In yet another embodiment, each discrete region comprises a unique first biomolecule. In yet, still another embodiment, at least a portion of the first biomolecule is derived from the same protein.

Preferably, the mass of the modified target probe or compound is measured in a time-of-flight mass spectrometer by ionization through laser desorption pulses.

These and other objects and features of the invention will be more fully appreciated when the following detailed description of the invention is read in conjunction with the accompanying drawings. 5

### BRIEF DESCRIPTION OF THE DRAWINGS

FIG. 1 is a perspective view of a substrate constructed in accordance with one embodiment of the present invention;

FIG. 2A is a map showing the features and relevant restriction sites of plasmid pET-17b containing a sequence of interest and a C-terminal coiled-coil domain;

FIG. 2B is a map showing the features and relevant restriction sites of plasmid pET-17b containing a sequence of interest and a N-terminal coiled-coil domain;

FIG. 3 is a cross-sectional view taken in the direction of arrows 3-3 in FIG. 1 of a multisubunit complex composition containing one of a plurality of subunit polypeptides in each well constructed in accordance with one embodiment of the invention;

FIGS. 4A-4D illustrate the steps in the assembly of individual subunit polypeptides of a multisubunit complex in one embodiment of the present invention;

FIGS. 5A-5B illustrate the steps in the assembly of a multisubunit complex by adding the subunit polypeptides as a mixture;

FIG. 6 is a view of a substrate constructed in accordance with one embodiment of the invention;

FIG. 7 shows an example of a K coil plate plus E-GFP (top panel) along with controls: K coil plate plus GFP, and standard MS plate with only E-GFP (middle and bottom panels, respectively);

FIG. 8 illustrates a biofunction chip with enzymatic functions according to one embodiment of the invention;

FIG. 9 illustrates on-chip phosphorylation of AbI Protein Tyrosine Kinase according to one embodiment of the invention

FIG. 10 illustrates on-chip phosphorylation in rabbit reticulocyte extract with various substrate concentrations (0, 10, 30, 100, and 300 nM) added according to one embodiment of the invention;

FIG. 11 shows in vitro AbI PTK substrate expression and control MALDI MS spectra;

FIG. 12 shows graphs depicting phosphorylated signal versus phosphorylated substrate;

FIG. 13 shows the reproducibility of the spectra in the same scale and in different scales normalized to the phosphorylated peaks;

FIG. 14 illustrates the application of the invention to screening test compounds for their ability to inhibit substrate phosphorylation; ATTP, an ATP analog, inhibits at increasing concentrations of ATTP;

FIG. 15 shows the inhibition of phosphorylation by the ATP analog Genistein; and

FIG. 16 shows the inhibition of phosphorylation by the substrate analog Erbstain.

### DETAILED DESCRIPTION OF THE INVENTION

I. Definitions

Unless otherwise indicated, all technical and scientific terms used herein have the same meaning as they would to one skilled in the art of the present invention. Practitioners are particularly directed to Sambrook et al (2001) “Molecular Cloning: A Laboratory Manual” Cold Spring H arb or Press, 3rd Ed., and and Ausubel, F. M., et al. (1993) in Current Protocols in Molecular Biology, for definitions and terms of the art. It is to be understood that this invent ion is not limited to the particular methodology, protocols, and reagents described, as these may vary. All publications and patents cited herein are expressly incorporated herein by reference for the purpose of describing and disclosing com positions and methodologies which might be used in connection with the invention.

The terms “protein,” “polypeptide,” or “peptide” as used herein refers to a biopolymer composed of amino acid or amino acid analog subunits, typically some or all of the 20 common L-amino acids found in biological proteins, linked by peptide inter subunit linkages, or other inter subunit linkages. The protein has a primary structure represented by its subunit sequence, and may have secondary helical or pleat structures, as well as overall three-dimensional structure. Although “protein” commonly refers to a relatively large polypeptide, e.g., containing 100 or more amino acids, and “peptide” to smaller polypeptides, the terms are used interchangeably herein. That is, the term protein may refer to a larger polypeptide, as well as to a smaller peptide, and vice versa.

The term “small molecule” includes a compound or molecular complex, either synthetic, naturally derived, or partially synthetic, and which preferably has a molecular weight of less than 5,000 Daltons. More preferably, a small molecule has a molecular weight of between 100 and 1,500 Daltons.

The term “biomolecule” refers to a molecule, synthesized artificially or by a biological organism, that is water soluble and typically charged in the pH range of from about 6 to about 9. Preferably, the term biomolecule includes proteins, glycoproteins, natural and synthetic peptides, alkaloids, polysaccharides, nucleic acid molecules, small molecules and the like. More preferably, the term biomolecule refers to proteins.

The term “conservative substitution” is used in reference to proteins or peptides to reflect amino acid substitutions that do not substantially alter the activity (specificity or binding affinity) of the molecule. Typically, conservative amino acid substitutions involve substitution of one amino acid for another amino acid with similar chemical properties (e.g., charge or hydrophobicity). The following six groups each contain amino acids that are typical conservative substitutions for one another:

i. Alanine (A), Serine (S), Threonine (T);

ii. Aspartic acid (D), Glutamic acid (E);

iii. Asparagine (N), Glutamine (Q);

iv. Arginine (R), Lysine (K);

v. Isoleucine (1), Leucine (L), Methionine (M), Valine (V); and

vi. Phenylalanine (F), Tyrosine (Y), Tryptophan (W).

The term “nucleic acid molecule” includes RNA, DNA and cDNA molecules. It will be understood that, as a result of the degeneracy of the genetic code, a multitude of nucleotide sequences encoding given peptides such as E-coil and K-coil peptides may be produced.

A “heterologous” nucleic acid construct or sequence has a portion of the sequence which is not native to the cell in which it is expressed. Heterologous, with respect to a control sequence refers to a control sequence (i.e. promoter or enhancer) that does not function in nature to regulate the same gene the expression of which it is currently regulating. Generally, heterologous nucleic acid sequences are not endogenous to the cell or part of the genome in which they are present, and have been added to the cell, by infection, transfection, microinjection, electroporation, or the like. A “heterologous” nucleic acid construct may contain a control sequence/DNA coding sequence combination that is the same as, or different from a control sequence/DNA coding sequence combination found in the native cell.

As used herein, the term “vector” refers to a nucleic acid construct designed for transfer between different host cells. An “expression vector” refers to a vector that has the ability to incorporate and express heterologous DNA fragments in a foreign cell. Many prokaryotic and eukaryotic expression vectors are commercially available. Selection of appropriate expression vectors is within the knowledge of those skilled in the art.

As used herein, an “expression cassette” or “expression vector” is a nucleic acid construct generated recombinantly or synthetically, with a series of specified nucleic acid elements that permit transcription of a particular nucleic acid in a target cell or in vitro. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter.

As used herein, the term “plasmid” refers to a circular double-stranded (ds) DNA construct used as a cloning vector, and which forms an extrachromosomal self-replicating genetic element in many bacteria and some eukaryotes.

As used herein, the term “selectable marker-encoding nucleotide sequence” refers to a nucleotide sequence which is capable of expression in host cells and where expression of the selectable marker confers to cells containing the expressed gene the ability to grow in the presence of a corresponding selective agent.

As used herein, the terms “promoter” and “transcription initiator” refer to a nucleic acid sequence that functions to direct transcription of a downstream gene. The promoter will generally be appropriate to the host cell in which the target gene is being expressed. The promoter together with other transcriptional and translational regulatory nucleic acid sequences (also termed “control sequences”) are necessary to express a given gene. In general, the transcriptional and translational regulatory sequences include, but are not limited to, promoter sequences, ribosomal binding sites, transcriptional start and stop sequences, translational start and stop sequences, and enhancer or activator sequences.

“Chimeric gene” or “heterologous nucleic acid construct”, as defined herein refers to a non-native gene (i.e., one that has been introduced into a host) that may be composed of parts of different genes, including regulatory elements. A chimeric gene construct for transformation of a host cell is typically composed of a transcriptional regulatory region (promoter) operably linked to a heterologous protein coding sequence, or, in a selectable marker chimeric gene, to a selectable marker gene encoding a protein conferring antibiotic resistance to transformed host cells. A typical chimeric gene of the present invention, for transformation into a host cell, includes a transcriptional regulatory region that is constitutive or inducible, a protein coding sequence, and a terminator sequence. A chimeric gene construct may also include a second DNA sequence encoding a signal peptide if secretion of the target protein is desired.

A nucleic acid is “operably linked” when it is placed into a functional relationship with another nucleic acid sequence. For example, DNA encoding a secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the term “gene” means the segment of DNA involved in producing a polypeptide chain, that may or may not include regions preceding and following the coding region, e.g. 5′ untranslated (5′ UTR) or “leader” sequences and 3′ UTR or “trailer” sequences, as well as intervening sequences (introns) between individual coding segments (exons).

As used herein, “recombinant” includes reference to a cell or vector, that has been modified by the introduction of a heterologous nucleic acid sequence or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention.

The term “introduced” in the context of inserting a nucleic acid sequence into a cell, means “transfection”, or “transformation” or “transduction” and includes reference to the incorporation of a nucleic acid sequence into a eukaryotic or prokaryotic cell where the nucleic acid sequence may be incorporated into the genome of the cell (for example, chromosome, plasmid, plastid, or mitochondrial DNA), converted into an autonomous replicon, or transiently expressed (for example, transfected mRNA).

As used herein, the term “expression” refers to the process by which a polypeptide is produced based on the nucleic acid sequence of a gene. The process includes both transcription and translation.

The term “signal sequence” refers to a sequence of amino acids at the N-terminal portion of a protein which facilitates the secretion of the mature form of the protein outside the cell. The mature form of the extracellular protein lacks the signal sequence which is cleaved off during the secretion process.

By the term “host cell” is meant a cell that contains a vector and supports the replication, or transcription and translation (expression) of the expression construct. Host cells for use in the present invention can be prokaryotic cells, such as E. coil or eukaryotic cells such as yeast, plant, insect, amphibian, or mammalian cells.

As used herein, the terms “active” and “biologically active” refer to a biological activity associated with a particular target protein, such as the enzymatic activity. It follows that the biological activity of a given protein refers to any biological activity typically attributed to that protein by those of skill in the art.

II. Method of the Invention

The invention includes, in one aspect, a method of protein interaction on a solid support. It has been discovered that a protein conjugate bound to a solid support through interactions with a coil-forming peptide can be used to identify other polypeptides to which it binds, to determine the kinetics of self assembly and to screen drugs.

There are several features of the invention that when used in combination with various analysis methods as described below, provide a number of advantages. These features include: (i) having the binding moiety produced as part of a protein of interest; (ii) allowing single-well in vitro transcription and translation of a protein of interest and immobilization of the translated protein; (iii) the stoichiometry of the binding agent, and its placement on the protein of interest (at either the C- or N-terminus) can be accurately controlled; (iv) the binding event has little effect on the presentation and/or activity of the immobilized and presented protein; and (v) the binding can disrupted by an acidic matrix when practiced in combination with MALDI mass spectrometry. Considered below are the steps in practicing the invention.

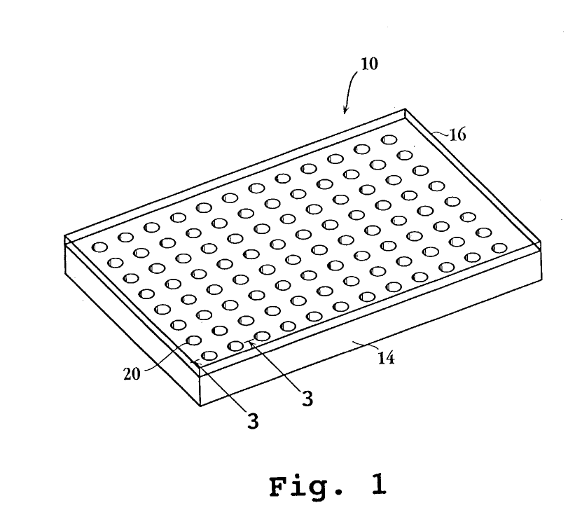

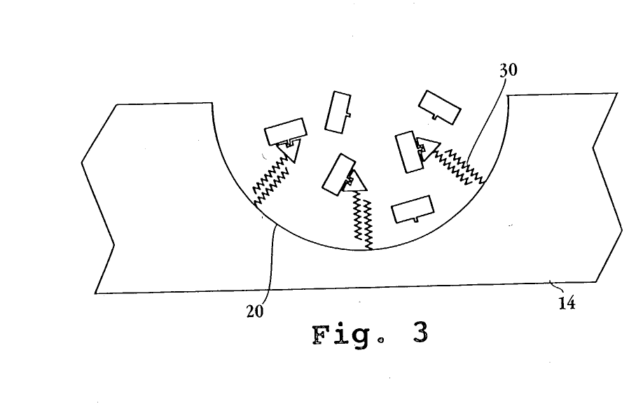

FIG. 1 is a plan view of a substrate 14, and optionally, a covering 16 which may be transparent and is attached to the substrate. The substrate includes a plurality of discrete wells 20. As shown in FIG. 3, each well 20 in substrate 14 is functionalized with a first coil-forming peptide 30 having a selected charge and being capable of interacting with a second, oppositely charged coil-forming peptide to form a stable α-helical coiled-coil heterodimer. The two oppositely charged peptides spontaneously self-assemble into a heterodimer complex. The interaction of coiled-coil heterodimers is further described in Section II.C. below.

A. Forming the Conjugate

The method employs a chimeric polypeptide conjugate that includes a coil-forming peptide and a first subunit polypeptide. Preferably, the first subunit polypeptide is one of a plurality of subunit polypeptides in a multisubunit complex. The coil-forming peptide has an ability to interact with a second, oppositely charged coil-forming peptide to form a stable α-helical coiled-coil heterodimer. The chimeric polypeptide conjugate can be formed by a number of methods, including recombinant methods and protein synthesis and ligation methods as described below.

A1. Recombinant Methods

The chimeric polypeptide conjugate of the invention may be produced by recombinant protein expression methods. For example, an expression vector that includes a DNA sequence encoding the coil-forming peptide operably linked to a DNA sequence encoding the first subunit polypeptide may be employed. Conventional molecular biology methods for constructing expression vectors are known to those of skill in the art.

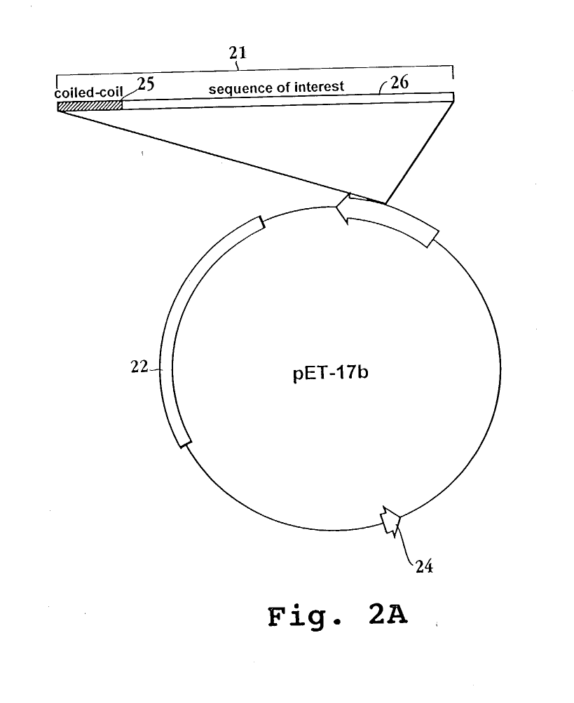

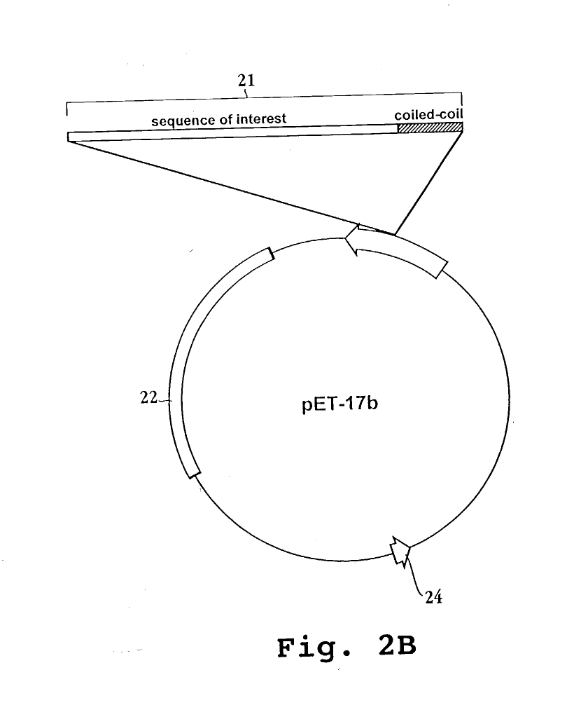

FIGS. 2A and 2B illustrate expression vectors for use in the present invention comprising a coding sequence 21, designed for operation in an in vitro or in vivo expression system, with companion sequences 22 and 24 upstream and downstream from the coding sequence. The coding sequence may have the coiled-coil region 25 at the C-terminus of the sequence of interest 26 as shown in FIG. 2A. Alternatively, the coiled-coil region 25 of the coding sequence 21 may be at the N-terminus of the 20 sequence of interest 26, as shown in FIG. 2B.

The expression vectors may be transferred to a host cell by conventional molecular biology methods to produce a transfected host cell for the expression of the chimeric polypeptide conjugate. The transfected cell is cultured by conventional cell culture techniques so as to produce the conjugate. Alternatively, the expression vector may be transcribed and translated in vitro to produce the conjugate. The coding sequence for the polypeptides may comprise cDNA or genomic DNA or both.

The host cell used to express the recombinant polypeptide of the invention may be either a bacterial cell such as Escherichia coli, or preferably a eukaryotic cell. In one embodiment, a mammalian cell such as a chinese hamster ovary cell is used. The choice of expression vector is dependent upon the choice of host cell, and may be selected so as to have the desired expression and regulatory characteristics in the selected host cell.

The general methods for construction of the vector of the invention, transfection of cells to produce the host cell of the invention, in vitro transcription and translation, and culture of cells to produce the conjugate of the invention are all conventional molecular biology methods. Likewise, once produced, the conjugate of the invention may be purified by standard procedures of the art, including cross-flow filtration, ammonium sulphate precipitation, affinity column chromatography, gel electrophoresis and the like.

Other exemplary methods for forming the chimeric polypeptide conjugate include PCR amplification or mRNA isolation and primer ligation as described in co-owned U.S. Patent Application No. 60/314,333, filed Aug. 22, 2001, and U.S. Patent application Ser. No. 10/225,788, filed Aug. 22, 2002, each of which is expressly incorporated by reference in its entirety herein.

A2. Protein Synthesis and Ligation Methods

The chimeric polypeptide conjugate may be constructed by protein synthesis and ligation methods. The first subunit polypeptide, which may be one of a plurality of subunit polypeptides in a multisubunit complex can be synthesized or derivatized after synthesis, to provide the requisite attachment function for ligating the coil-forming peptide. In general, most conjugation methods do not disrupt the coil-forming activity of the coil-forming peptide or the activity and/or conformation of the first subunit polypeptide.

For example, enzymatic ligation of cloned or synthetic peptide segments can allow relatively short peptide fragments to be joined to produce larger peptide fragments, polypeptides or whole protein domains (Abrahmsen et al., 1991). Alternatively, native chemical ligation of synthetic peptides can be utilized to synthetically construct large peptides or polypeptides from shorter peptide fragments. This method consists of a two step chemical reaction (Dawson et al., 1994). The first step is the chemoselective reaction of an unprotected synthetic peptide-[proportional]-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (Clark-Lewis et al., 1987; Clark-Lewis et al., 1994; Clark-Lewis et al., 1991; and Rajarathnam et al., 1994).

Alternatively, unprotected peptide segments can be chemically linked where the bond formed between the peptide segments as a result of the chemical ligation is an unnatural or non-peptide bond (Schnolzer et al., 1992). This technique has been used to synthesize analogs of protein domains as well as large amounts of relatively pure proteins with full biological activity (deLisle Milton et al., 1992).

B. Immobilizing the First Coil-forming Peptide

As noted, one coil-forming peptide is anchored to a substrate. Suitable methods for immobilizing peptides on solid substrates include ionic, hydrophobic, and covalent interactions and the like. An exemplary method for immobilizing a peptide on a solid support is described in U.S. Pat. No. 5,071,909, which is incorporated by reference in its entirety herein.

The solid support comprises regions that are spatially discrete and addressable or identifiable. Each region comprises a coiled-coil peptide 30 bound thereto. In one embodiment, the regions are separated from one another by any physical barrier that is resistant to the passage of liquids. In another embodiment, a substrate such as a MALDI-MS plate is etched out to have discrete, shallow wells. Alternatively, a substrate may comprise regions with no separations or wells, for example a flat surface, and individual regions may be further defined by overlaying a structure (e.g., a piece of plastic or glass) which delineates the separate regions. A variety of techniques known in the art may be used to generate an array of discrete regions. For example, patches of an organic thinfilm may be generated by microstamping (U.S. Pat. Nos. 5,512,131 and 5,731,152), microfluidics printing, inkjet printers, or manually with multi- or single-channel pipettes. The relative orientation of the regions can take any of a variety of forms including, but not limited to, parallel or perpendicular arrays within a square or rectangle or other surface, radially extending arrays within a circular substrate, or linear arrays.

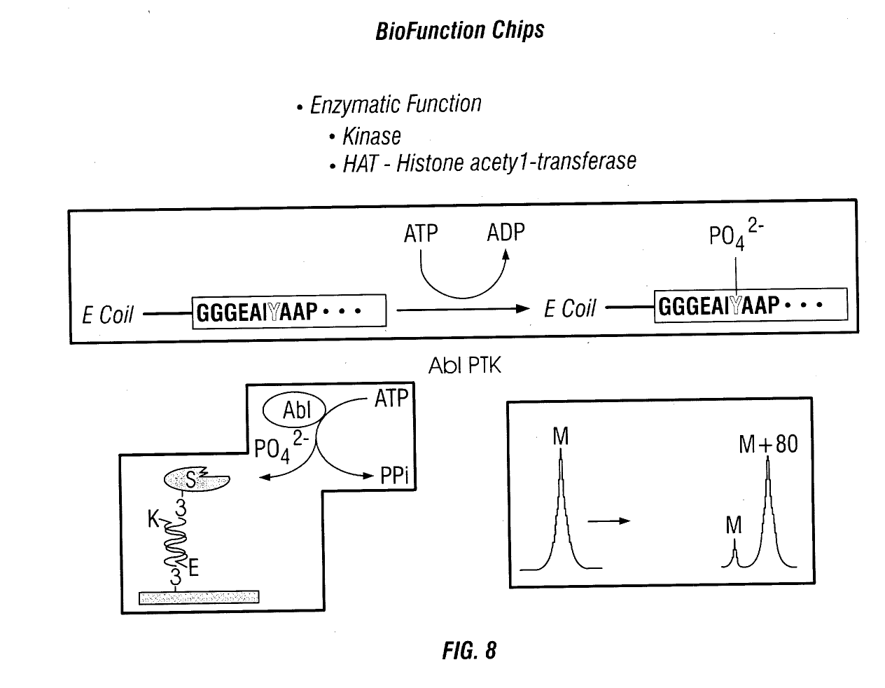

The array itself may range from the standard microtiter plate format (e.g., 24, 48, 96, 384, or 1536 wells), to a small microarray containing hundreds of spots within 1 to several cm². Thus, in one embodiment, the invention comprises a substrate having at least 2 discrete regions on an array. Preferably, the substrate has at least 10 discrete regions on one array. More preferably, the substrate comprises at least 10² discrete regions on one array. Even more preferably, the substrate comprises at least 10³ discrete regions on one array. Even more preferably, the substrate comprises at least 10⁴ discrete regions on one array. Two exemplary arrays are illustrated in FIG. 8.

The number of bound coil-forming peptides in a region can be at least two, preferably between about 5 and about 10000, more preferably between about 10 and about 1000, and most preferably between about 50 and about 500. In one embodiment, the coil-forming peptides are bound at a density of between about 1×10² to 1×10¹⁵ coil-forming peptides/mm². In another embodiment, the coil-forming peptides are bound at a density of between about 1×10⁵ to 1×10¹² coil-forming peptides/mm². In yet another embodiment, the coil-forming peptides are bound at a density of between about 1×10¹⁰ to 1×10¹¹ coil-forming peptides/mm². In yet, still another embodiment, the coil-forming peptides are bound at a density of less than about 8.5×10¹⁰ coil-forming peptides/mm².

The distances between regions vary depending on the layout of the array. For example, in an embodiment, two or more regions are arranged in a section of an array comprising a total area of about 1 cm² or less. In a preferred embodiment, 5 or more regions are arranged in a section of an array comprising a total area of about 1 cm² or less. An exemplary embodiment of an array showing dimensions of and between regions in an array is shown in FIG. 8.

A “solid surface,” “solid support,” “solid substrate,” or “chip” as used herein, refers to any material which is insoluble, or can be made insoluble by a subsequent reaction. The solid surface can be chosen for its intrinsic ability to attract and immobilize the first coil-forming peptide. Alternatively, the solid phase can retain an attachment molecule which has the ability to attract and immobilize the first coil-forming peptide. The attachment molecule can include a charged substance that is oppositely charged with respect to the first coil-forming peptide itself or to a charged substance conjugated to the first coil-forming peptide. As yet another alternative, the attachment molecule may be any specific binding member which is immobilized upon or attached to the solid phase and which has the ability to immobilize the first coil-forming peptide through a specific binding reaction. The solid surface thus can be a plastic, derivatized plastic, magnetic or non-magnetic metal, glass or silicon surface of a test tube, microtiter well, sheet, bead, microparticle, chip, and other configurations known to those of ordinary skill in the art. Exemplary solid supports are described in U.S. Pat. No. 5,591,440, which is expressly incorporated by reference in its entirety herein.

In one embodiment of the invention, the solid surface is comprised of a semiconductor material composition which has photoluminescence properties when made porous. Such semiconductor material compositions may include, without limitation, cadmium, copper oxide, germanium, gallium, gallium arsenide, selenium, silicon, silicon carbide, silicon dioxide, silicon gallium phosphide and combinations thereof. The selected semiconductor material compositions may also incorporate a dopant, including, for example, without limitation, erbium, boron, phosphorous, copper, phosphors of the lanthanides series, including ytterbium, holmium and thulium, and combinations thereof. Also, the selected semiconductor material compositions may be processed with another compound, including, for example, without limitation, a halogen, such as bromine, to modify the emission wavelength (Bressers et al., 1996). In a preferred embodiment, the solid support or solid surface is a plate suitable for use in a Matrix Assisted Laser Desorption Ionization-Time of Flight mass spectrometer (MALDI-MS), as described below.

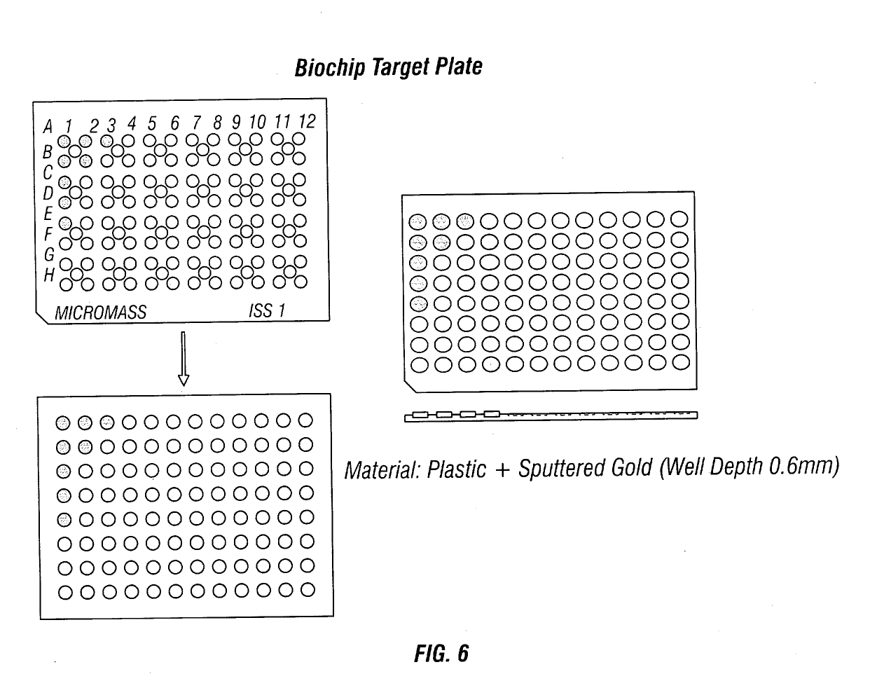

The substrate may include a coating. The coating may be formed on, or applied to, the binding surface. For example, in one embodiment of the invention, the coating is a metal film, such as gold, as illustrated in FIG. 6. Additional suitable metals which may be used for coating include, but are not limited to, platinum, silver, copper, zinc, nickel, and cobalt. Additionally, commercial metal-like substances may be employed such as TALON metal affinity resin and the like. Coatings may be applied by electron-beam evaporation or physical/chemical vapor deposition. In order to promote stable and efficient immobilization of the coil-forming peptide on the solid substrate, the surface of the substrate may be first stablilized against uncontrolled oxidation. One such stabilization procedure is oxidation using, for example, thermal oxidation (Petrova-Koch et al., 1992), chemical oxidation (Lee et al., 1995), and ozone oxidation processes (Janshoff et al., 1998). These processes generate reactive hydroxyl groups. The coating may cover the entire substrate, or may be limited to regions comprising an associated binding surface.

C. Binding the Conjugate to the Surface

When a first coil-forming peptide and a second coil-forming peptide are mixed together under conditions favoring the formation of α-helical coiled-coil heterodimers, they interact to form a two-subunit α-helical coiled-coil heterodimeric complex. Peptides in an α-helical coiled-coil conformation interact with one another in a characteristic manner that is determined by the primary sequence of each peptide. The tertiary structure of an α-helix is such that seven amino acid residues in the primary sequence correspond to approximately two turns of the α-helix. Accordingly, a primary amino acid sequence giving rise to an α-helical conformation may be broken down into units of seven residues each, termed heptads. The heterodimer-subunit peptides are composed of a series of heptads in tandem. When the sequence of a heptad is repeated in a particular heterodimer-subunit peptide, the heptad may be referred to as a “heptad repeat”, or simply “repeat”.

A first coil-forming peptide and second coil-forming peptide may assemble into a heterodimer coiled-coil helix (coiled-coil heterodimer) in either parallel or antiparallel configurations. In a parallel configuration, the two heterodimer-subunit peptide helixes are aligned such that they have the same orientation (amino-terminal to carboxyl-terminal). In an antiparallel configuration, the helixes are arranged such that the amino-terminal end of one helix is aligned with the carboxyl-terminal end of the other helix, and vice versa. Such heterodimer subunits are described in PCT patent application WO 95/31480 “Heterodimer Polypeptide Immunogen Carrier Composition and Method”, publication date Nov. 23, 1995, which is incorporated herein by reference in its entirety. Additional sequences and subunits are described in U.S. Pat. Nos. 6,478,939; 6,461,490; 6,165,335; 6,130,037; and 5,955,379; each of which is incorporated by reference herein in its entirety.

Exemplary subunits are referred to herein as K-coils, referring to positively charged subunits whose charge is provided dominantly by lysine residues, and E coils, referring to negatively charged subunits whose charge is provided dominantly by glutamic acid residues. Preferred examples from the above-mentioned application include SEQ ID NOS: 1-2.

Heterodimer-subunit peptides designed in accordance with the guidance presented in the above-referenced application typically show a preference for assembling in a parallel orientation versus an antiparallel orientation. For example, the exemplary peptides identified by SEQ ID NO:3 and SEQ ID NO:4 form parallel-configuration heterodimers as do other peptide sequences (as discussed in the WO 95/31480 application). When attaching a protein of interest to a first coil-forming peptide it is generally desirable to attach the protein of interest at the distal end of the heterodimer. In particular, where the heterodimer forms a parallel configuration, the second coil-forming peptide is preferably anchored to the substrate surface at its C-terminus, and the protein of interest is conjugated to the first coil-forming peptide at its N-terminus.

As noted, one of the two subunit peptides in the heterodimer is anchored to the substrate, and the other peptide contains a protein of interest which is one of a plurality of subunit polypeptides in a multisubunit complex. In both cases, the peptide can be synthesized or derivatized after synthesis, to provide the requisite attachment function. In general, most conjugating methods do not disrupt the coil-forming activity of either of the coil-forming peptide, nor do such conjugations disrupt the activity of the conjugated protein of interest.

Considering the modification of the first coil-forming peptide, the peptide may be synthesized at either its N- or C-terminus to carry additional terminal peptides that can function as a spacer between the substrate surface and the helical-forming part of the peptide. Alternatively, the first coil-forming peptide can be attached to the substrate surface through a high-affinity binding reaction such as between a biotin moiety carried on the peptide and an avidin molecule covalently attached to the surface.

The protein of interest may be synthesized, as noted above, by either solid-state, PCR, or recombinant methods, in vivo or in vitro to include the protein of interest at the end of the second coil-forming peptide that will orient distally in the assembled heterodimer. In forming the conjugate through solid-state methods, the protein of interest is preferably covalently attached to the N-terminal amino acid residue, or to one of the residues facing the exposed face of the heterodimer. Preferred coupling groups are the thiol groups of cysteine residues, which are easily modified by standard methods. Other useful coupling groups include the thioester of methionine, the imidazolyl group of histidine, the guanidinyl group of arginine, the phenolic group of tyrosine and the indolyl group of tryptophan. These coupling groups can be derivatized using reaction conditions known to those skilled in the art.

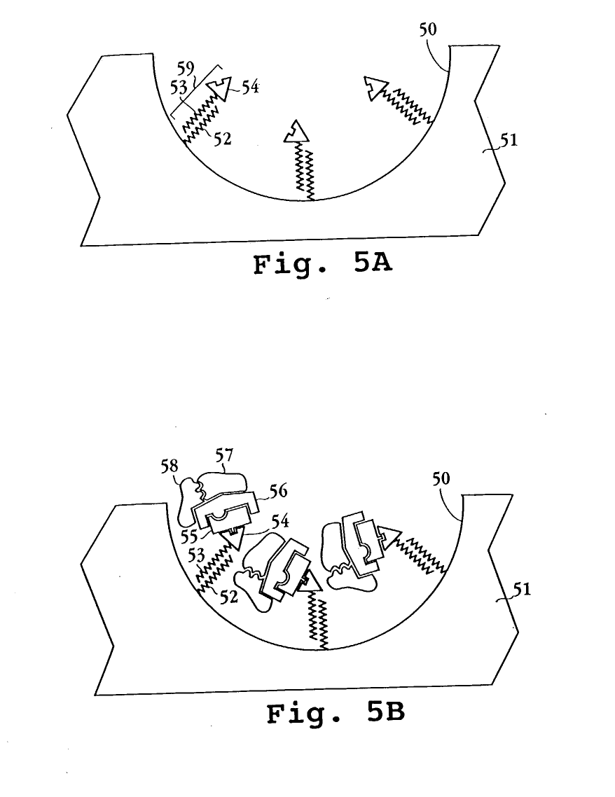

To bind the target protein-second coil-forming peptide conjugate 59 (FIG. 5A) to the surface-immobilized first coil-forming peptide 52, the two peptides are contacted under conditions that favor heterodimer formation. An exemplary medium favoring coiled-coil heterodimer formation is a physiologically-compatible aqueous solution typically having a pH of between about 6 and about 8 and a salt concentration of between about 50 mM and about 500 mM. Preferably, the salt concentration is between about 100 mM and about 200 mM. An exemplary benign medium has the following composition: 50 mM potassium phosphate, 100 mM KCl, pH 7. Equally effective media may be made by substituting, for example, sodium phosphate for potassium phosphate and/or NaCl for KCl. Heterodimers may form under conditions outside the above pH and salt range, medium, but some of the molecular interactions and relative stability of heterodimers vs. homodimers may differ from characteristics detailed above. For example, ionic interactions between the ionic groups that tend to stabilize heterodimers may break down at low or high pH values due to the protonation of, for example, Glu side chains at acidic pH, or the deprotonation of, for example, Lys side chains at basic pH. Such effects of low and high pH values on coiled-coil heterodimer formation may be overcome, however, by increasing salt concentration.

Increasing the salt concentration can neutralize the stabilizing ionic attractions or suppress the destabilizing ionic repulsions. Certain salts have greater efficacy at neutralizing the ionic interactions. For example, in the case of the K-coil peptide, a 1 M or greater concentration of CIO⁴⁻ anions is required to induce maximal α-helical structure, whereas a 3M or greater concentration of Cl ions is required for the same effect. The effects of high salt on coiled-coil formation at low and high pH also show that interhelical ionic attractions are not essential for helix formation, but rather, control whether a coiled-coil tends to form as a heterodimer versus a homodimer. The E- and K-coil peptides can also be conjugated to proteins of interest or other biomolecules as in Example 2 of co-owned U.S. application Ser. No. 09/654,191 (Attorney Docket #: 54800-8015.US01), which is expressly incorporated by reference herein in its entirety.

As noted above, the K and E coils may be made synthetically or by recombinant DNA techniques. Those of skill in the art will recognize that minor sequence variations may be made without compromising the ability of the coils to dimerize specifically or significantly affect their binding affinity for each other. It is also recognized that short amino acid linkers such as multi-glycines may be added at either termini of either coil, and will ordinarily not affect the formation of the coiled coil structure. Furthermore, chemically active groups such as bifunctional cross-linkers may be added to the termini of these coils to facilitate conjugation of the coil to a protein, peptide or other biomolecule for presentation.

D. Additional Multicomplex Subunits

According to one aspect of the invention, the first subunit polypeptide of the conjugate is one of a plurality of subunit polypeptides in a multisubunit complex. Following binding of the conjugate to the solid surface through α-helical coiled-coil heterodimer formation with an immobilized peptide, additional subunits of the complex are added under conditions effective to promote self-assembly of the subunit complex on the support.

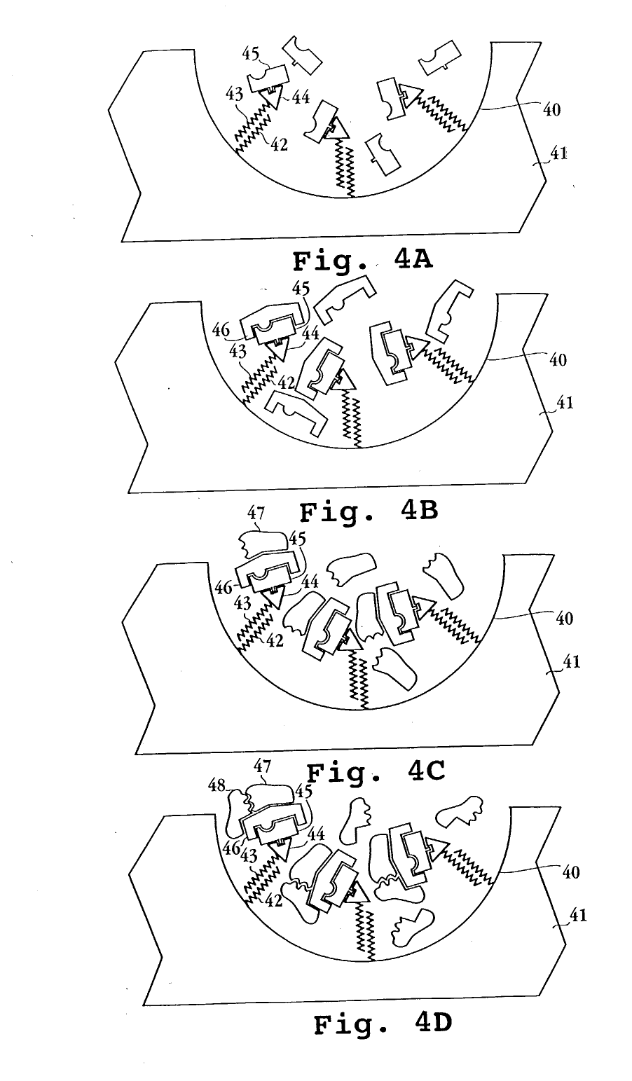

In one embodiment, the multisubunit complex is assembled by adding the subunit polypeptides individually. Referring to FIGS. 4A-4D, each well 40 in substrate 41 has a first coil-forming peptide 42 therein. The conjugate, which comprises the second coil-forming peptide 43 and the first subunit polypeptide 44, is bound to the solid surface through coiled-coil heterodimer formation. A second subunit polypeptide 45, which is one of the plurality of subunit polypeptides in the multisubunit complex, is added to the well under conditions effective to promote self-assembly of the first and second subunit polypeptides 44 and 45 as shown in FIG. 4A. Likewise, a third subunit polypeptide 46, which is one of the plurality of subunit polypeptides in the multisubunit complex, is added to well 40 under conditions effective to promote self-assembly of the first, second and third subunit polypeptides 44, 45 and 46 as shown in FIG. 4B. This may be continued, as illustrated in FIGS. 4C-4D, until the desired subunit polypeptides 47 and 48 of the multisubunit complex are assembled on the solid surface.

In another embodiment, the multiprotein complex is assembled by adding the subunit polypeptides as a mixture as illustrated in FIGS. 5A-5B. The wells in the substrate may be filled with a solution that contains these subunit polypeptides. As shown in FIG. 5A, each well 50 in substrate 51 has a first coil-forming peptide 52 therein. The conjugate 59, which comprises the second coil-forming peptide 53 and the first subunit polypeptide 54, is bound to the solid surface through coiled-coil heterodimer formation. A mixture of subunit polypeptides 55, 56, 57, 58 of the multiprotein complex is added to well 50 under conditions effective to promote self-assembly of the mixture of polypeptides with the first subunit polypeptide 54, as illustrated in FIG. 5B. Following self-assembly of the polypeptides in the mixture, the wells may be washed to remove unbound components.

Conditions effective to promote self-assembly of the subunit polypeptides in the multisubunit complex are conventional and described throughout the literature, and will vary with the nature of the subunit polypeptides and/or the multisubunit complex. See e.g., U.S. Pat. Nos. 6,294,363 and 6,083,708 which are incorporated by reference herein. Various buffer systems and conditions described in the literature and known to those of skill in the art may be employed. In one embodiment of the invention, the presented protein, peptide or other biomolecule and the complex that is formed can be membrane-associated when the appropriate surfactant or surfactants known to those of skill in the art are added.

E. Exemplary Multisubunit Complexes

Nuclear hormone receptors are grouped into a large superfamily and are thought to be evolutionarily derived from a common ancestor. Seven subfamilies of mammalian nuclear receptors exist. Class I consists of the following thyroid hormone receptor, retinoic acid receptor, vitamin D receptor, peroxisome proliferator activated receptor, pregnane X receptor, constitutive androstane receptor, liver X receptor, farnesoid X receptor, reverse ErbA, retinoid Z receptor/retinoic acid-related orphan receptor and the ubiquitous receptor. Class II consists of: retinoid X receptor, chicken ovalbumin upstream promoter transcription factor, hepatocyte nuclear factor 4, tailles-related receptor, photoreceptor-specific nuclear receptor and testis receptor. Class III includes: glucocorticoid receptor, androgen receptor, progesterone receptor, estrogen receptor and estrogen-related receptor. NGF-induced clone B is a class IV nuclear receptor; steroidogenic factor 1 and Fushi Tarazu factor 1 are class V receptors; germ cell nuclear factor is a class VI receptor; and, small heterodimeric partner and dosage-sensitive sex reversal are class 0 receptors. Reviewed in Aranda and Pascual, 2001.

Ligands for some of these receptors have been identified, showing that products of lipid metabolism such as fatty acids, prostaglandins, or cholesterol derivatives can regulate gene expression by binding to nuclear receptors. These nuclear receptors bind to hormone response elements as monomers, homodimers, or RXR heterodimers to DNA. Ligands may play a role in dimerization and binding to DNA (Ribeiro, 1992). A number of proteins interact with these receptors, including general transcription factors. As with other transcriptional regulatory proteins, one aspect of the mechanisms by which nuclear receptors affect the rate of RNA polymerase II-directed transcription likely involves the interaction of receptors with components of the transcription preinitiation complex. This interaction may be direct, or it may occur indirectly through the action of bridging factors (Schulman, 1995). Sequence-specific transcription factors (Castillo, 1999), coactivators and corepressors (Cavailles, 1995) also have been found to interact with these nuclear receptors.

Voltage-dependent calcium channels mediate the entry of calcium into neurons and other excitable cells and play important roles in a variety of neuronal functions, including membrane excitability, neurotransmitter release, and gene expression. Calcium channels are multisubunit complexes with the channel activity mainly mediated by the pore-forming subunit; however, additional subunits act as accessory proteins that regulate channel activity (Catterall, 1995).

Ubiquitin-mediated protein degradation is a highly selective process that is achieved through the concerted action of a versatile set of enzymes (Hershko and Ciechanover, 1998; Varshavsky, 1997). A single E1 enzyme (ubiquitin activating enzyme) is responsible for activation of the small protein ubiquitin, which is then passed on via trans-acetylation to several E2 enzymes (ubiquitin conjugating enzyme). Each E2 may collaborate with several different E3 proteins in creating a protein-ubiquitin conjugate. The E3s, referred to as ubiquitin-protein ligases, confer specificity to the system and share a common property: substrate recognition and binding. Whereas the E2 proteins bear a significant homology to each other, the E3s many of which are associated with large multisubunit complexes, form a highly heterogenous group. Within these complexes the specific task of individual subunits is not always clear (Yamano et al., 1998; Zachariae and Nasmyth, 1999). Moreover, the composition of the complex is not necessarily static and may be subject to regulatory processes associated with the functional status of the cell (Fang et al., 1998; Zachariae et al., 1998). Only a few E3s have been characterized in detail and there is only scant information regarding mammalian E3s. Among the latter, one of the better-defined E3s is SCF (beta-TrCP/E3RS), a recently identified E3 complex that targets plkappaBalpha and beta-catenin for degradation (reviewed in (Karin and Ben-Neriah, 2000; Maniatis, 1999; Polakis, 1999)).

Additional multisubunit complexes are known in the art and described in the literature, and include without limitation, the nuclear pore complex, the ribosome complex, the 26S proteosome complex, the F0F1 ATPase complex, DNA polymerase, and components of the transcriptional initiation complex, which includes RNA polymerase II (which is composed of at least 12 subunits) and TFIID, TFIIB, TFIIA, TFIIF, TFIIE, and TFIIH (reviewed in Wilson, et al. 1996). Also contemplated are complexes comprising one or more nucleic acid molecules.

F. Identifying Bound Polypeptides

The protein interaction methods and biofunction chips of the present invention allow for the rapid analysis of large numbers of samples in a short amount of time, thereby reducing the cost of analysis. Thus, the invention contemplates coupling high throughput detection systems to identify the multisubunit complexes and products of the biofunction chips.

There are a number of different types of detection systems suitable to assay the multisubunit complexes and other products of the invention. Such systems include, but are not limited to, fluorescence, absorbance/transmittance, radiometric counting, measurement of electronic effects upon exposure to a compound oranalyte, luminescence, ultraviolet visible light, collision induced dissociation (CID), CCD cameras, electron and three dimensional microscopy. Other techniques are known to those of skill in the art. For example, laser induced fluorescence (LIF) detection methods for the analyses of combinatorial arrays and biochip formats are described in Ideue, S. et al. (2000).

In a preferred embodiment, a mass spectrometry detection system is employed. A useful mass spectrometry detection system can be any of various formats, including ionization (I) techniques such as matrix assisted laser desorption (MALDI), continuous or pulsed electrospray (ESI), ionspray, thermospray, or massive cluster impact (MCI) as described in U.S. Pat. No. 6,225,061, which is expressly incorporated by reference herein. Such ion sources can be matched conveniently with a detection format, including linear or reflectron time-of-flight (TOF), single or multiple quadruple, single or multiple magnetic sector, Fourier transform ion cyclotron resonance (FTICR), ion trap, and combinations thereof to yield a hybrid detector, for example, ion-trap/time-of-flight. For ionization, numerous matrix/wavelength combinations (MALDI) or solvent combinations (ESI) can be employed. MALDI-TOF mass spectrometry, including delayed extraction MALDI-TOF mass spectrometry is particularly useful as a detection system. See, for example, International PCT application No. WO98/20019; and Whittal et al., (1998).

Using parallel sampling techniques, TOF mass spectrometry may be used for the detailed characterization of hundreds of molecules in a sample mixture at each discrete location within the array. These systems enable extremely rapid analysis and high levels of selectivity.

In a preferred embodiment of the method of the invention, the following steps are included: (i) preparing a protein of interest that has an E or K coil terminal segment; (ii) adding the protein to a well having the complementary K or E coil; (iii) immobilizing the protein in the well by coil-coil binding; (iv) washing non-bound components from the well, leaving the bound protein; and (v) analyzing the presented protein.

When the method of the invention is practiced in combination with MALDI mass spectrometry, as described above, the entire method may be performed in individual wells on a MALDI palte. In a preferred embodiment, the following steps are included: (a) adding a solution of light absorbing matrix to the substrate immediately prior to mass spectrometry analysis, and allowing the solution to dry on the substrate (the addition of the matrix solution is effective to dissociate the protein from the substrate, such that the dried material on the substrate includes a mixture of crystalline matrix and free, or non-immobilized, protein); and (b) irradiating the protein/matrix mixture with a laser beam, causing the matrix to undergo rapid energy release, imparting the explosion energy to the dissociated protein.

In this embodiment, the protein-protein dissociation preferably occurs following addition of an acidic matrix (pH 2-5) to the well. As evidence that the protein release can occur prior to mass spectrometry analysis, the inventors have performed a series of experiments showing that the matrix solution causes dissociation. In the first experiment, matrix was added to a well that had the protein of interest bound to the well through the coil-coil interaction discussed above. MALDI analysis resulted in a quantitative profile of the protein of interest analyzed. In the second experiment, matrix was added to a well that had the protein of interest bound to the well as in the first experiment. However, prior to analysis, the matrix solution and any dissociated proteins were removed from the well and spotted in a second well. MALDI analysis of the second well, containing the removed matrix solution and dissociated proteins, showed a quantitative profile of the protein of interest that was substantially equal to the profile obtained in the first experiment. Thus, the matrux solution dissociates the coil-coil interaction prior to energy desorption and ionization by the laser.

III. Utility

As indicated above, the method is readily adapted to reconstituting a multi-protein complex. The method is illustrated in FIGS. 4A-4D, which show a coil-forming peptide 42 interacting with a conjugate comprising a second coil-forming peptide 43 and the first subunit polypeptide 44 to form a multiprotein complex. The method is also adaptable to determining the kinetics and/or order of subunit assembly by analyzing the bound subunits at different times following addition of the subunits to the regions.

The method of the invention is also useful in identifying and/or isolating unknown binding subunits. In this embodiment, a coiled coil heterodimer is used to present a ligand or biomolecule, e.g., a protein, peptide, nucleic acid, small molecule, carbohydrate and the like, and the biomolecule is used to further bind its natural, mutated or synthetic receptor or receptors. Thus, in this embodiment, a multisubunit complex consists of the receptor and its ligand. In one embodiment, the coiled coil presented biomolecule grabs one other protein, peptide or biomolecule. This complex is then submitted for scientific studies such as screening for agonists/antagonists, functional assays such as enzymatic activity detection, or other assays known to those of skill in the art.

In a preferred embodiment, the method is useful for identifying target eceptors that bind to orphan secreted biomolecules, the binding affinity of the biomolecule for its receptor and compounds that may modulate biomolecule-receptor binding. Thus, in one embodiment of the invention, the first subunit polypeptide is a biomolecule that binds specifically to the receptor, e.g. a nuclear hormone receptor. The receptor is added to the region under conditions effective to promote self-assembly of the receptor and biomolecule, e.g., orphan protein. Following self-assembly, the region may be washed to remove unbound components. After washing, the receptor may be identified using one or more of the methods described above. Alternatively, the first subunit polypeptide is a receptor, and the biomolecule, which may be a ligand or other binding partner of the receptor, e.g. RXR nuclear hormone receptor subunit, is added to the region under conditions effective to promote self-assembly of the receptor and biomolecule. Following washing, the biomolecule or biomolecules (ligand or receptor binding partner or partners or all components of the complex) are identified as previously described. Thus, the presented receptor is used to bind natural, mutated or known or unknown interacting biomolecules.

This is also termed “mining.”

In another preferred embodiment, the immobilized first coil-forming peptide is fused with a single copy of a nuclear hormone receptor. A suitably prepared E coil-nuclear hormone receptor, which may be either a homo- or hetero-dimer subunit is added to the surface. E/K dimer formation preferentially creates a dimeric nuclear hormone receptor complex. The complex is the used in the mining process. The site on the nuclear hormone receptor where the K coil is fused may be identified in such a manner as to promote formation of a dimeric nuclear hormone receptor as the E/K coiled coil is formed, similar to the nuclear hormone receptor observed in nature.

In yet another preferred embodiment, the presentation surface is first functionalized with a first coil-forming peptide. An E coil fused with a first subunit (e.g., a biomolecule such as another protein, nucleic acid molecule or antibody) that recognizes the dimeric form of the nuclear hormone receptor is added. The nuclear hormone receptor is then added to the surface for and binds to the first subunit under conditions effective to promote self-assembly of the subunit and nuclear hormone receptor complex on the solid surface.

The method is also readily adapted to screening a plurality of test compounds for their ability to modulate, e.g. enhance or inhibit, the binding of one or more subunit polypeptides to one or more other subunit polypeptides in a multisubunit complex, e.g., the 26S proteosome complex. The purpose of such an assay is to test a plurality of compounds for their ability to alter the extent of inter-subunit binding, for example, as part of a drug-discovery program to find a compound capable of interfering with subunit-subunit binding.

FIGS. 4A-4D illustrate the assay in the absence of test compounds, where binding of subunits 44 and 45 (FIG. 4A), and subsequent subunits 46, 47 and 48 (FIGS. 4B-4D) are not effected by a test compound. In this embodiment, the coiled coil presented protein, peptide or other biomolecule grabs or binds one other protein, peptide or other biomolecule, and the secondary molecule recruits a tertiary molecule, and the tertiary moiety then recruits others that may bind to the existing complex. This is termed “sequential complex assembly.” The formed complex is then submitted for conventional scientific studies known to those of skill in the art. In another embodiment, as illustrated in FIGS. 5A-5B, the coiled coil presented protein, peptide or biomolecule 54 binds a complex 55, 56, 57, 58 or multiple complexes that already exist in the immediate surroundings or environment or region. This is termed “non-sequential complex assembly.” Binding of multicomplexes may or may not be cooperative or sequential in nature. The assembled complex or multicomplexes are then analyzed as described above.

In one embodiment of the invention that is not shown, a test compound is first added to the region 40, which may bind to subunit 44., Binding of subunit 45 to subunit 44 may be inhibited, either partially or completely, depending on the relative concentrations of subunits 45 and test compound, and the relative affinities of the two for subunit 44.

In a preferred embodiment, a plurality of multisubunit complexes, e.g. in a microtiter-plate format, are each mixed with a different test compound or with a different concentration of the same test compound. Following binding, the reaction mixtures are detected. Where each sample contains a different test compound, the relative effect of each of a plurality of such compound on subunit-subunit binding can be determined. Where each sample contains a different concentration of the same test compound, the relative binding affinity and range of effective compound concentrations can be determined.

In another embodiment, the method is suitable to assess enzymatic activities if the first subunit polypeptide is, e.g. a substrate and/or the product formed can be distinguished by mass spectrometry, fluorescence, radiometric counting, optical and other biochemical techniques.

In another embodiment, the method is adapted for use as a biosensor device wherein an electrochemical or optical detection scheme is used to assess the progress or result of the experiments. Biosensors are described in, e.g., U.S. Pat. Nos. 6,300,141; 6,165,335; 6,130,037; 6,107,080; and 5,955,379, each of which is expressly incorporated by reference herein.

In another embodiment of the invention, an in vivo mining process may be employed. In one embodiment, a coding sequence that includes a first nucleic acid sequence which encodes a first coil-forming peptide, e.g. an E coil, and a nuclear hormone receptor gene is transfected into selected normal and/or diseased host cells. The host cells are treated with agents such as hormones or drugs. The cells are lysed and the E coil tagged nuclear hormone receptor complex is harvested using a K coil functionalized matrix such as a column, membrane, or biochip.

IV. Protein Presentation

The invention includes, in another aspect, a method for carrying out the presentation of a plurality of target proteins as described in above-mentioned co-owned U.S. Patent Application No. 60/314,333, filed Aug. 22, 2001, which is expressly incorporated by reference herein. In general, a selected different-sequence nucleic acid molecule, from a plurality of different-sequence nucleic acid molecules, is added to each of a plurality of wells in a substrate. Each well in the substrate has a first coil-forming peptide therein. Each different-sequence nucleic acid molecule has two portions: a common-sequence capture portion encoding a second coil-forming peptide, and a different-sequence target portion encoding a target protein.

The wells in the substrate are filled with a solution that contains protein synthesis components capable of expressing the different-sequence nucleic acid molecules under selected protein-synthesis conditions. The different-sequence nucleic acid molecules are then expressed. The target proteins expressed in each well bind to the well through coil-coil heterodimer formation with the substrate-bound coil forming peptide and are thus presented for analysis in the well in captured form. The wells can then be washed to remove unbound components. These presented proteins may then be used for ligand-receptor screening as discussed above, structure/function studies, e.g. alanine scanning mutagenesis, antibody panels and 2-hybrid studies.

In one embodiment, each different-sequence target portion is a different cDNA molecule selected from a library of cDNA molecules. Following expression, the presented proteins in each well have a different sequence. The target proteins expressed in each well bind to the well through coil-coil heterodimer formation with the substrate-bound coil forming peptide and are thus presented for analysis in the well in captured form. Each protein is representative of the cDNA library. The presented proteins can then be screened against one or more drugs to identify the proteins that interact with a selected drug.

In another embodiment, a protein that has been mutated in a different region is placed in each well such that on a given substrate each well contains a different mutant of the same protein. For example, a 96 well plate would have 96 different mutations of the same protein. A protein or drug is used to screen the plate for high affinity binding. Mass spectrometry could then be used to identify where the mutation resides that is responsible for the increased binding affinity of the protein or drug.

In yet another embodiment, each different-sequence target portion is encoded by the same DNA molecule. Following expression and protein binding, the presented proteins in each well are all identical. The presented proteins can then be screened against a panel of different compounds to identify a drug that interacts with the presented protein. In one embodiment, a chemical library is subdivided into pools and then each pool is added to each well. Mass spectrometry is used to identify a compound or pool of compounds that bind specifically to the presented protein. In another embodiment a DNA library is subdivided into pools and then each pool is added to each well. Mass spectrometry is used to identify a specific DNA binding sequence for the presented protein. In yet another embodiment, the presented protein is an enzyme or enzyme variant that is presented in each well. A library of potential enzyme substrates are added to the wells, and mass spectrometry is used to identify product formation in the well; or in the case of an inhibitor, tight binding molecules.

The presented proteins can be used to monitor biochemical reactions as described above, such as, e.g., interactions of proteins, nucleic acids, small molecules, or the like. For example, the efficiency of specificity of interactions between antigens and antibodies; or of receptors (such as purified receptors or receptors bound to cell membranes) and their ligands, agonists or antagonists; and enzymes (such as proteases or kinases) and their substrates, or increases or decreases in the amount of substrate converted to a product; as well as many others. Such biochemical assays can be used to characterize properties of the target protein, or as the basis of a screening assay. For example, to screen samples for the presence of particular proteases Xa and VIIa), the samples can be assayed on combinations in which the target proteins are individual proteases. If a fluorogenic substrate specific for a particular presented protease binds to the protease and is cleaved, the substrate will fluoresce, usually as a result, e.g. of cleavage and separation between two energy transfer pairs, and the signal can be detected. In another example, to screen samples for the presence of a particular kinase(s) (e.g., Src, tyrosine kinase, or ZAP70), samples containing one or more kinases of interest can be assayed on combinations in which the bound, presented polypeptides can be selectively phosphorylated by one of the kinases of interest.

Using art-recognized, routinely determinable conditions, samples can be incubated with the array of substrates, in an appropriate buffer and with the necessary cofactors, for an empirically determined period of time. After treating (e.g., washing) each reaction under empirically determined conditions to remove unbound and undesired components, the bound components can be detected by mass spectrometry.

In another embodiment, the presented proteins can be used to screen for agents which modulate the interaction of a presented protein and a given probe. An agent can modulate the protein/probe interaction by interacting directly or indirectly with either the probe, the protein or a complex formed by the protein plus the probe. The modulation can take a variety of forms, including, but not limited to an increase or decrease in the binding affinity of the protein for the probe, an increase or decrease in the rate at which the protein and probe bind, a competitive or non-competitive inhibition of the binding of the probe to the protein, or an increase or decrease in the activity of the probe or the protein which can, in some cases, lead to an increase or decrease in the probe/protein interaction. Such agents can be synthetic or naturally-occurring substances. Also, such agents can be employed in their unaltered state or as aggregates with other species; and they can be attached, covalently or noncovalently, to a binding member, either directly or via a specific binding substance.

From the foregoing, it can be seen how various objects and features of the invention are met.

### IV. EXAMPLES

The following examples further illustrate the invention described herein and are in no way intended to limit the scope of the invention.

### Example 1

Heterodimer protein technology (HPT) adds functionality to the biochip target plate. Well defined and spatially oriented protein layers are created. HPT uses a set of affinity peptides (K coils and E coils) to provide a method to tag, capture and present proteins on a surface. The K coil is a peptide made of 7-amino acid (SEQ ID NO: 5) repeats. In one embodiment, the K coil is 35 amino acids in length. It is positively charged, with no specific structure in solution. The E coil is a peptide made of 7-amino acid (SEQ ID NO: 6) repeats. In one embodiment, the E coil is 35 amino acids in length. It is negatively charged, and has no specific structure in solution.

Heterodimer E and K coils form unique helical and dimeric structures. The surface is prepared by adding 2-20 μm K coil for 30 minutes, rinsing with PBS-T, incubating in 1 mM cysteine for 30 minutes, and rinsing with PBS-T. In one embodiment, the E/K coil density is less than or equal to 8.5×10¹⁰ molecules/mm². This results in a stable surface of K coils that will not detach under MALDI MS and can be kept for an extended period of time, e.g. 1, 2, 8, 14 and 22 days.

The E coil is attached to a protein of interest by either conjugation chemistry or recombinant DNA technology. The E coil-protein is then added to the K coil surface and incubated for 15-30 minutes. The surface is then rinsed with PBS-T/water and analyzed using MS MALDI.

### Example 2

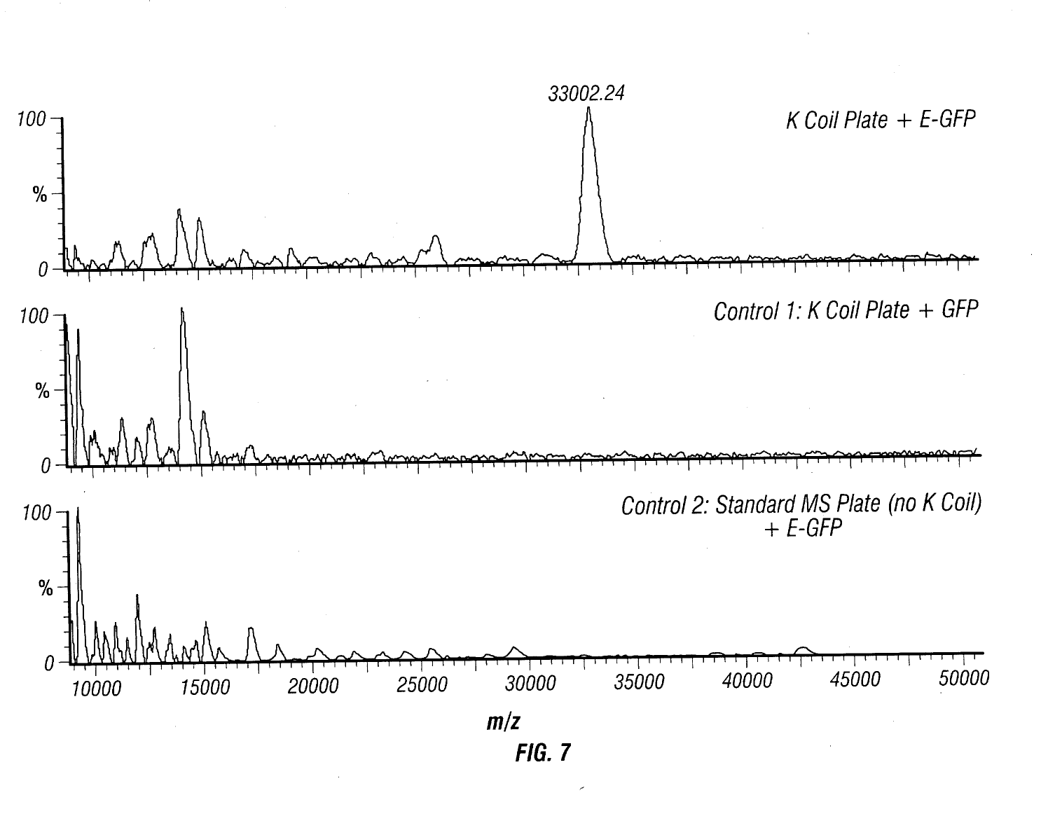

An on chip protein production procedure is performed wherein the expression, detection, isolation, characterization, and in vitro translation are all integrated onto one chip. Briefly, the K coils are bound to the surface of a well, a translation mix consisting of S30 bacterial extract rabbit reticulocyte lysate is added, an E fusion vector is added, the vector is translated for 30-60 minutes, and then washed and detected (15 min). FIG. 7 shows an example of a K coil plate plus E-GFP (top panel) along with controls K coil plate plus GFP, and standard MS plate with only E-GFP (middle and bottom panels, respectively).

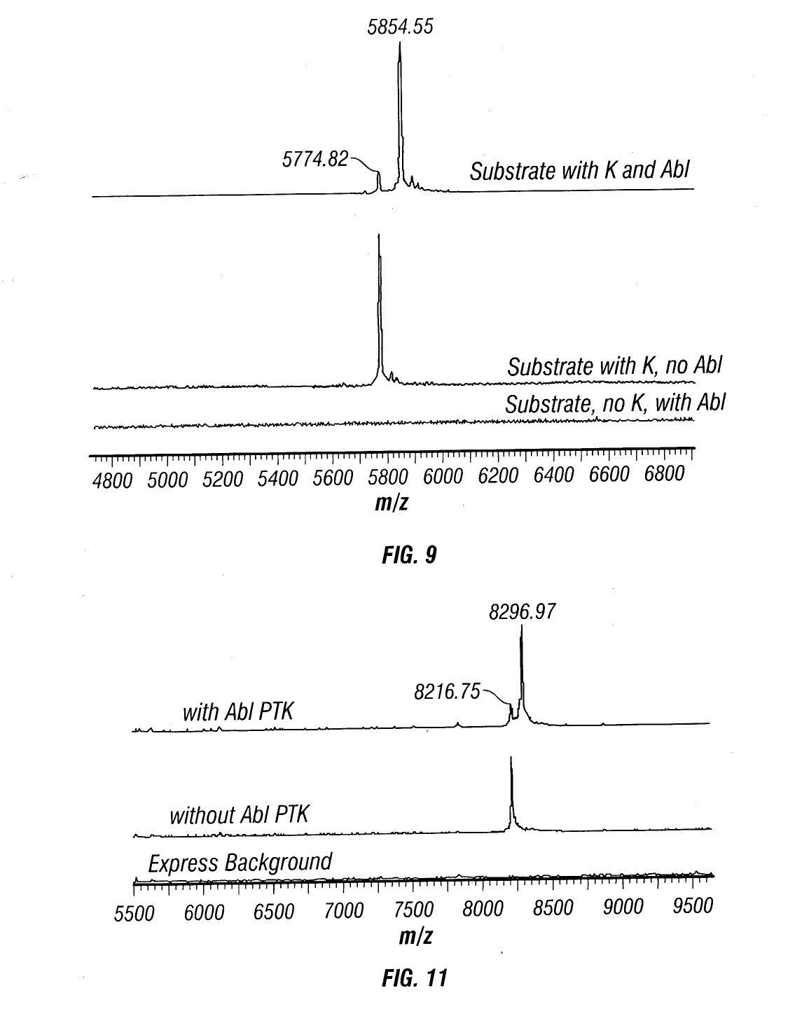

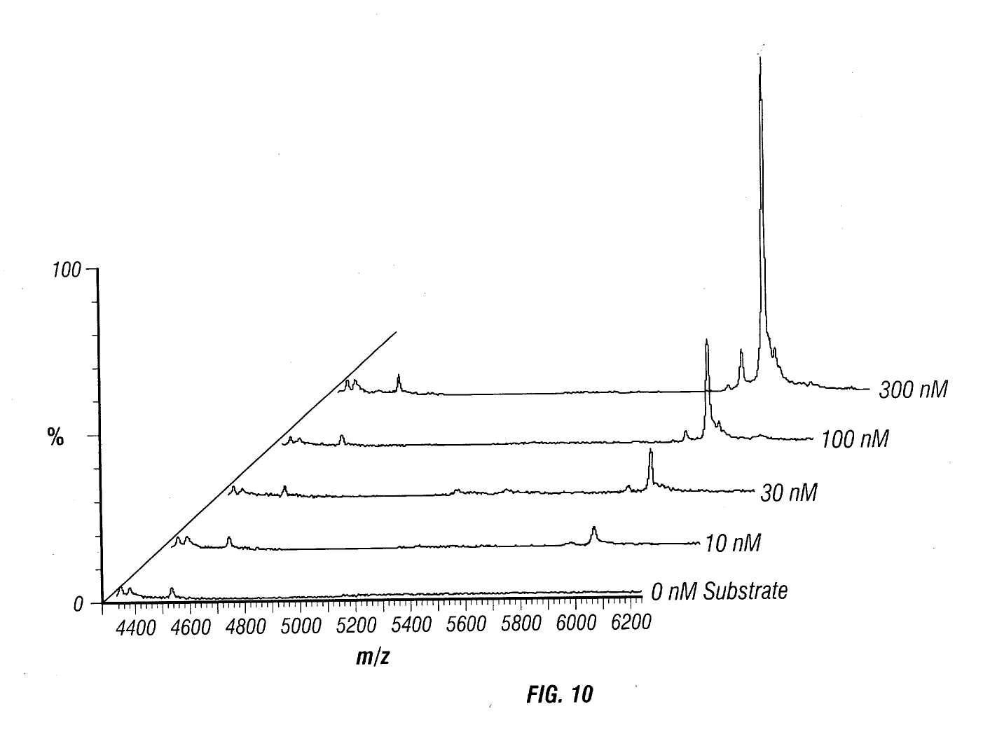

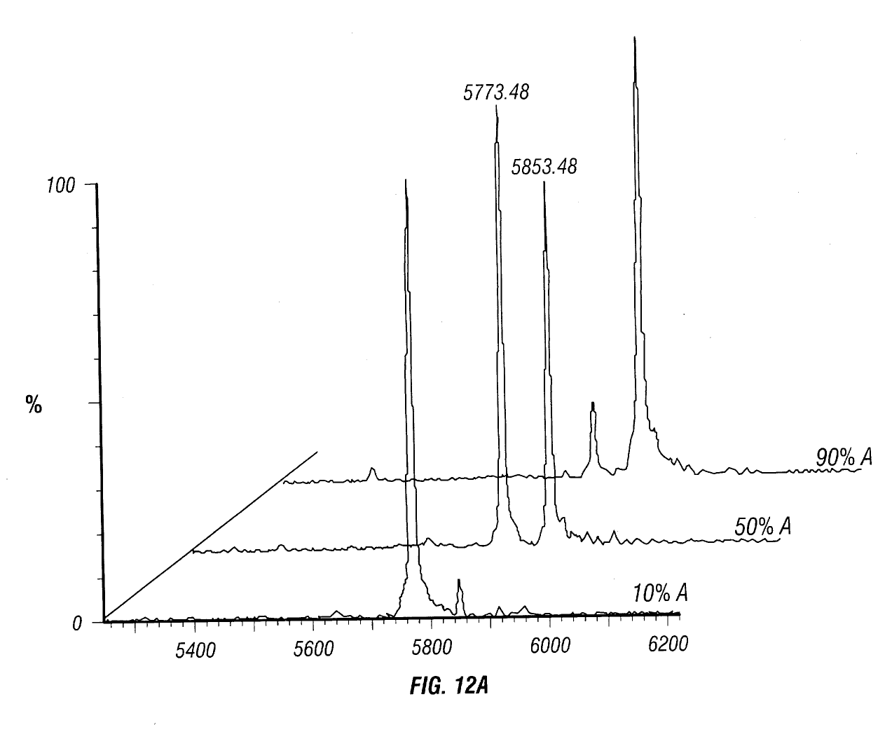

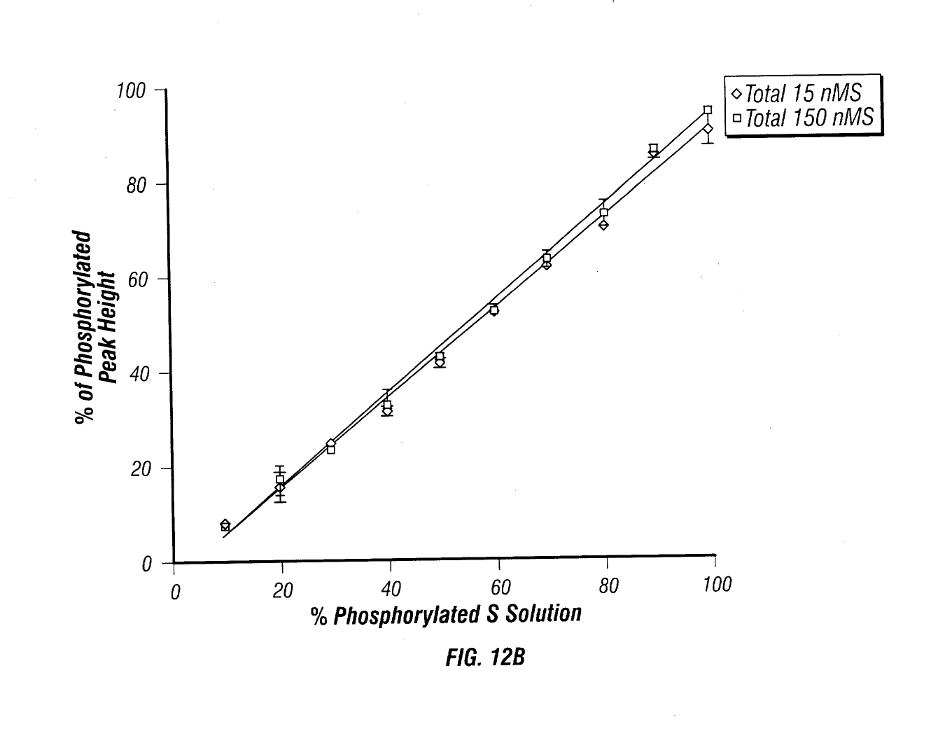

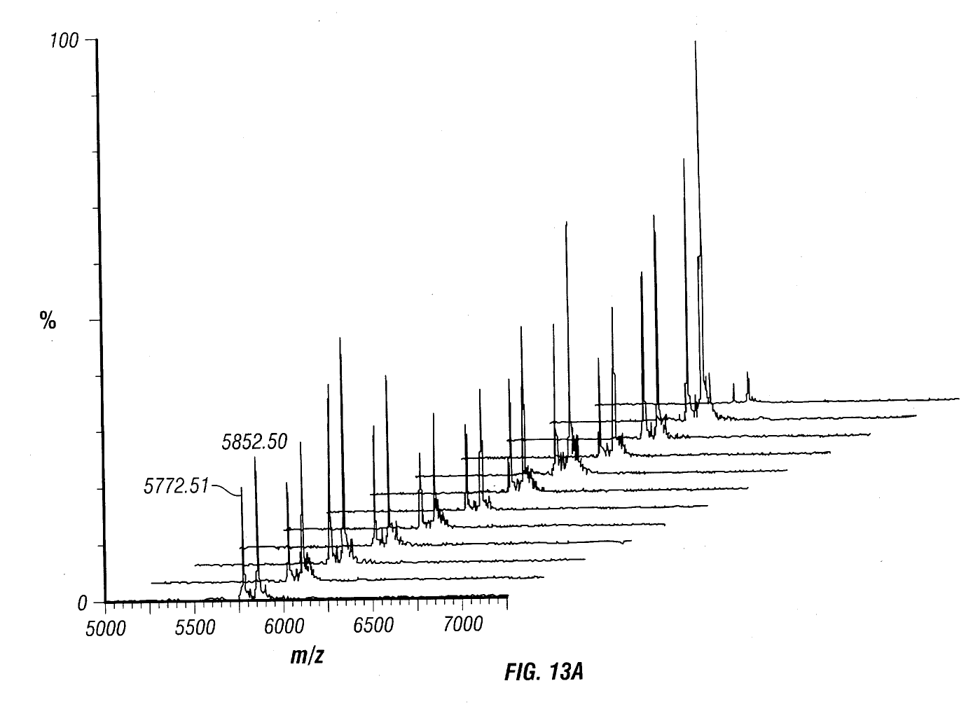

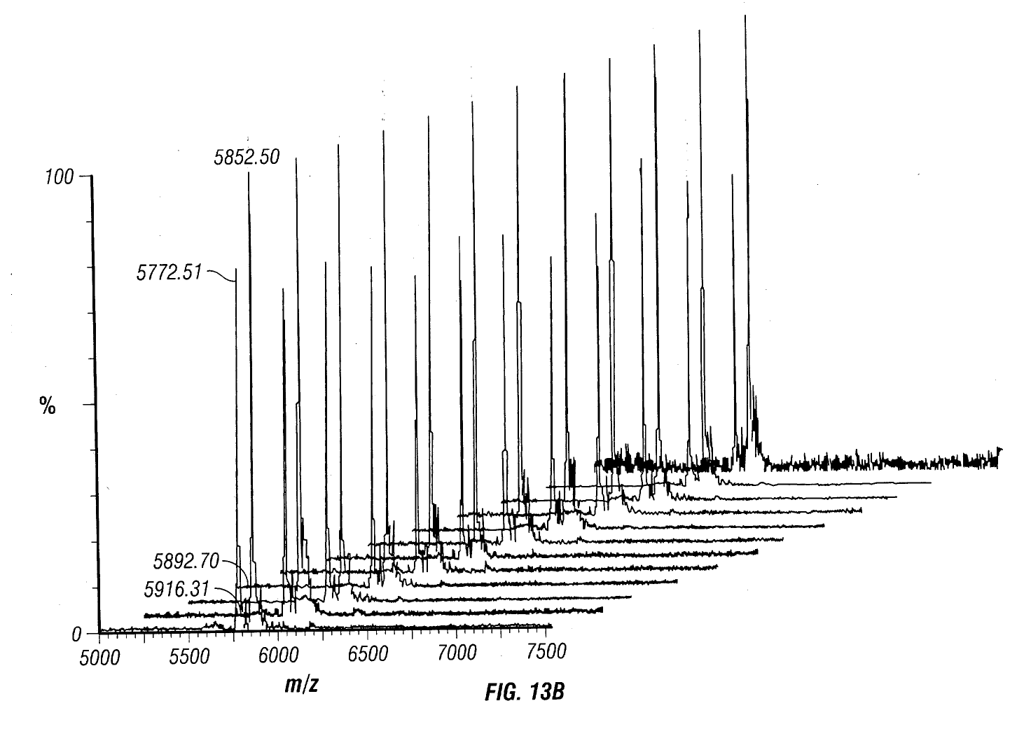

FIG. 8 illustrates a biofunction chip with enzymatic functions. On-chip phosphorylation of AbI Protein Tyrosine Kinase is shown in FIG. 9. The reaction components consisted of 0.4U/well of AbI at pH 7.5, 0.1 mM ATP, and 400 nM substrate. 4 μl were added per well, and the reaction was incubated at 23° C. for 90 minutes. On-chip phosphorylation in rabbit reticulocyte extract with various substrate concentrations (0, 10, 30, 100, and 300 nM) is shown in FIG. 10. In vitro AbI PTK substrate expression and control MALDI MS spectra are shown in FIG. 11. The phosphorylated signal versus the phosphorylated substrate is shown in FIG. 12. Reproducibility of the spectra in the same scale and in different scales normalized to the phosphorylated peaks are shown in FIG. 13. The percent phosphorylation is determined over time for 1 Unit of substrate per well versus 0.12 Unit substrate per well.

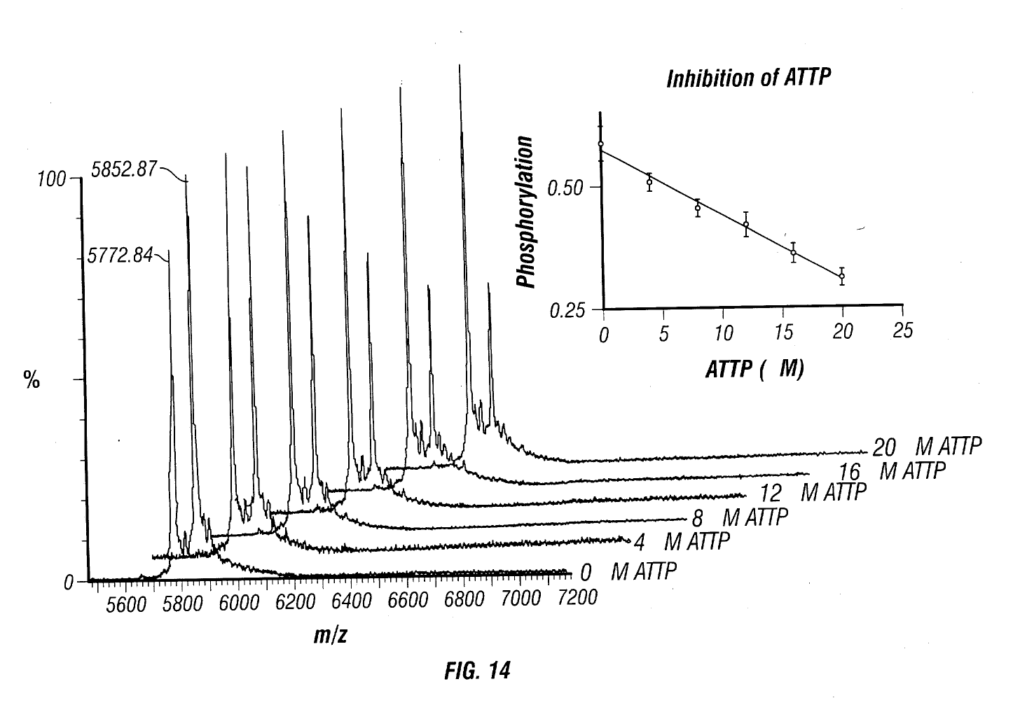

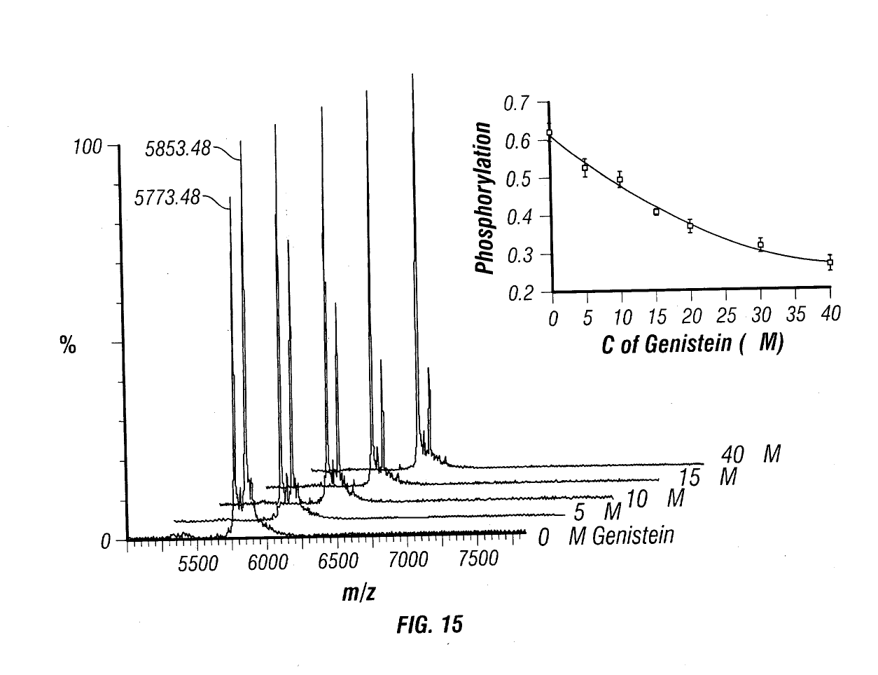

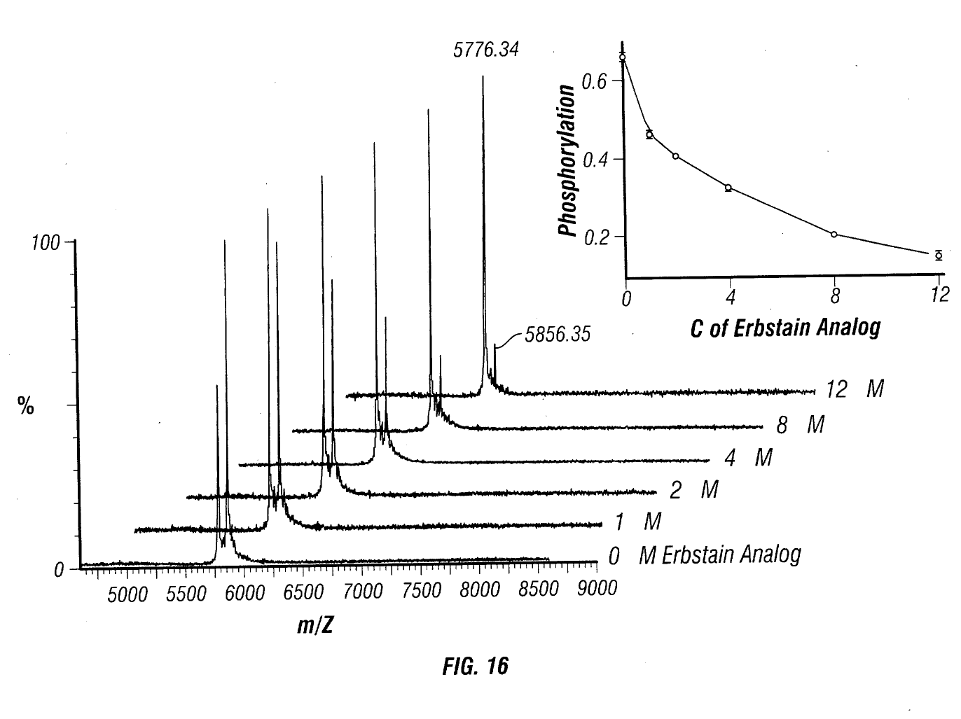

Screening assays for ATP analogs or substrate analogs capable of inhibiting phosphorylation were also tested. FIG. 14 shows the inhibition of phosphorylation by ATTP, an ATP analog, at increasing concentrations of ATTP. 250 nM substrate, 5 μM ATP, 20 U/well AbI TK, and 0, 4, 8, 12, 16, and 20 μM ATTP were incubated at 23° C. for 60 minutes. FIG. 15 shows the inhibition of phosphorylation by the ATP analog Genistein. 250 nM substrate, 5 μM ATP, 20 U/well AbI TK, and 0, 5, 10, 15 and 40 μM Genistein were incubated at 23° C. for 60 minutes. FIG. 16 shows the inhibition of phosphorylation by the substrate analog Erbstain. 250 nM substrate, 100 μM ATP, 0.4 Units/well AbI TK, and 0, 1, 2, 4, 8, and 12 μM Erbstain were incubated at 23° C. for 60 minutes.

The components of biofunction chips are useful for kinase studies, histone acetylation, DNA methylation and protease studies. Screening assays, e.g., may include an ATP analog-control ATP concentration (5 μm), and excess enzyme to achieve proper phosphorylation; and/or a substrate analog such that excess enzyme ATP concentrations (100 μM) are used and the enzyme is conrolled to achieve proper phosphorylation.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

It is claimed:

1. A protein interaction method, involving the steps of:
contacting a conjugate with a solid surface having an immobilized first coil-forming peptide characterized by a selected charge and an ability to interact with a second, oppositely charged coil-forming peptide to form a stable α-helical coiled-coil heterodimer, where the conjugate comprises
(a) the second, oppositely charged coil-forming peptide, and
(b) a first subunit polypeptide which is one of a plurality of subunit polypeptides in a multisubunit complex,
by said contacting, binding the conjugate to the solid surface, and
adding to the solid surface and bound first subunit polypeptide, one or more other subunits of the complex under conditions effective to promote self-assembly of the subunit complex on the solid surface.

2. The method of claim 1, wherein said first subunit polypeptide is a nuclear hormone receptor.

3. The method of claim 2, wherein said nuclear hormone receptor is selected from the group consisting of androgen receptors, thyroid receptors, estrogen receptors, vitamin D receptors, retinoic acid receptors, glucocorticoid receptors, and mineralocorticoid receptors.

4. The method of claim 1, for use in reconstituting a multiprotein complex of known formulation, where the other subunits are added individually.

5. The method of claim 1, for use in identifying unknown binding subunits, where the other subunits are added as a mixture.

6. The method of claim 1, for use in isolating a multisubunit protein complex from a host cell, wherein said other proteins in the multisubunit complex are pre-assembled and contained within said host cell, further comprising
lysing the host cells prior to adding the other proteins to the solid support; and
analyzing said multisubunit complex after adding the remaining subunits to determine the subunits constituting the multiprotein complex.

7. The method of claim 6, wherein said host cell is a diseased cell.

8. The method of claim 6, wherein said host cell is a normal cell.

9. The method of claim 6, wherein said host cell has been treated with an agent selected from the group consisting of hormones, ligands, and drugs.

10. The method of claim 1, for use in determining the kinetics and/or order of self assembly of a multisubunit protein complex, comprising
analyzing the subunits bound to the solid support at various times after the addition of the other subunits, and
determining the rate or order of subunit assembly of the protein complex.

11. The method of claim 1, for use in drug screening, further comprising contacting the solid surface with one or more chemical compounds under conditions effective to allow the compounds to bind to the self-assembled multisubunit protein complex;
washing the solid surface to remove unbound components; and
analyzing the complex to identify the bound compounds.

12. The method of claim 1, wherein said solid surface is a modified target plate suitable for MALDI mass spectrometry.

13. A method for carrying out the interaction of a plurality of multisubunit protein complexes, comprising
adding to each of a plurality of wells in a substrate, each well having a first coil-forming peptide therein, a selected one of a plurality of different-sequence subunit molecules, each having a common second coil-forming peptide capture portion and a different-sequence target protein portion selected from a plurality of subunits in a multisubunit protein complex;
contacting said wells with the remaining subunits from each of the multisubunit protein complexes under conditions effective to promote self-assembly of each of the complexes; and
washing the wells to remove unbound components.

14. A composition comprising
a first coil-forming peptide having a selected charge and capable of interacting with a second, oppositely charged coil-forming peptide to form a stable α-helical coiled-coil heterodimer immobilized on a solid surface;
a protein conjugate bound to the first coil-forming peptide, comprising
(a) the second, oppositely charged coil-forming peptide, and
(b) a first target subunit selected from a plurality of subunits in a multisubunit protein complex; and
other subunits of the complex assembled on the solid surface through protein interactions with the protein conjugate.

15. A biofunction chip for measuring the activity of a first or second biomolecule, comprising
a surface containing a plurality of spatially discrete regions, wherein each region is functionalized with a first coil-forming peptide having a selected charge and interacts with a second, oppositely charged coil-forming peptide to form a stable α-helical coiled-coil heterodimer; and
the first biomolecule attached to the distal end of the second coil-forming peptide,
whereby interaction of the first biomolecule with the second biomolecule is effective to modify the first or second biomolecule or both.

16. The biofunction chip of claim 15, wherein each region on the chip comprises
a plurality of first coil-forming peptides.

17. The biofunction chip of claim 15, wherein the first biomolecule is selected from the group consisting of proteins, glycoproteins, natural and synthetic peptides, alkaloids, polysaccharides, nucleic acid molecules, and small molecules.

18. The biofunction chip of claim 15, wherein the second biomolecule is selected from the group consisting of proteins, glycoproteins, natural and synthetic peptides, alkaloids, polysaccharides, nucleic acid molecules, and small molecules.

19. The biofunction chip of claim 15, wherein the first biomolecule is selected from the group consisting of a kinase substrate, a histone acetyl transferase substrate, and a protease substrate.

20. The biofunction chip of claim 17, wherein the first biomolecule is a nucleic acid molecule.

21. The biofunction chip of claim 20, wherein the nucleic acid molecule is modified by methylation.

22. The biofunction chip of claim 15, wherein each discrete region comprises a unique first biomolecule.

23. The biofunction chip of claim 15, wherein at least a portion of the first biomolecule is derived from the same protein.

24. The biofunction chip of claim 15, wherein the mass of the modified target probe or compound is measured in a time-of-flight mass spectrometer by ionization through laser desorption pulses.


SEQUENCE LISTING (7 sequences)

SEQ ID NO: 1 - protein, 35 aa, Artificial Sequence
  E coil peptide
EVSALEKEVSALEKEVSALEKEVSALEKEVSALEK

SEQ ID NO: 2 - protein, 35 aa, Artificial Sequence
  K coil peptide
KVSALKEKVSALKEKVSALKEKVSALKEKVSALKE

SEQ ID NO: 3 - protein, 35 aa, Artificial Sequence
  heterodimer-subunit peptide
EVEALQKEVSALEKEVSALECEVSALEKEVEALQK

SEQ ID NO: 4 - protein, 35 aa, Artificial Sequence
  heterodimer-subunit peptide
KVEALKKKVSALKEKVSALKCKVSALKEKVEALKK

SEQ ID NO: 5 - protein, 7 aa, Artificial Sequence
  K coil peptide
KVSALKE

SEQ ID NO: 6 - protein, 7 aa, Artificial Sequence
  E coil peptide
EVSALEK

SEQ ID NO: 7 - protein, 55 aa, Artificial Sequence
  VARIANT 20: Xaa = Any Amino Acid
KKKAFPAAYIAEGGGCGGGXEVSALEKEVSALEKEVSALEKEVSALEKEVSALEK